(19) United States
(12) Patent Application Publication
(10) Pub. No.: US 2025/0054571 A1
(43) Pub. Date: Feb. 13, 2025

(54) METHOD AND APPARATUS FOR PREDICTING RNA-PROTEIN INTERACTION, MEDIUM AND ELECTRONIC DEVICE

(71) Applicant: BOE TECHNOLOGY GROUP CO., LTD., Beijing (CN)

(72) Inventors: Chunhui ZHANG, Beijing (CN); Zhenzhong ZHANG, Beijing (CN)

(73) Assignee: BOE Technology Group Co., Ltd., Beijing (CN)

(21) Appl. No.: 17/915,391

(22) PCT Filed: Sep. 27, 2021
(86) PCT No.: PCT/CN2021/121089

§ 371 Date: Sep. 28, 2022

(51) Int. Cl. G16B 15/30; G16B 40/20

(52) U.S. Cl. CPC G16B 15/30; G16B 40/20

(57) ABSTRACT

A method for predicting an RNA-protein interaction, relates to the technical field of artificial intelligence. The method includes: acquiring an RNA-protein pair to be predicted; obtaining a sequence feature of the RNA-protein pair to be predicted by performing feature extraction on the RNA-protein pair to be predicted; obtaining an RNA sequence representation vector and a protein sequence representation vector in the RNA-protein pair to be predicted by vectorizing the RNA-protein pair to be predicted; obtaining respectively by using multiple interaction prediction models, multiple interaction prediction values of the RNA-protein pair to be predicted, based on the sequence feature of the RNA-protein pair to be predicted, the RNA sequence representation vector and the protein sequence representation vector in the RNA-protein pair to be predicted; and determining an interaction between the RNA and the protein according to the multiple interaction prediction values.

### CROSS REFERENCE

The present disclosure is a U.S. national phase application of International Application No. PCT/CN2021/121089, filed on Sep. 27, 2021, which is incorporated herein by reference in its entirety.

### SEQUENCE LISTING

The present application contains a Sequence Listing which has been submitted in ASCII format via EFS-Web and is hereby incorporated by reference in its entirety. The ASCII copy, created on May 15, 2024, is named 187002.00130_ST25 and is 629 bytes in size.

### TECHNICAL FIELD

The present disclosure relates to the technical field of artificial intelligence, and in particular, to a method and an apparatus for predicting an RNA-protein interaction, a computer-readable storage medium, and an electronic device.

### BACKGROUND

Noncoding RNA (ncRNA) is involved in many complex cellular processes, plays an important role in life processes such as alternative splicing, chromatin modification and epigenetics, and is closely related to many diseases. Studies have shown that most noncoding RNAs achieve their regulatory functions by interacting with protein. Therefore, studying the interaction between the noncoding RNA and the protein is of great significance for revealing the molecular mechanism of noncoding RNA in human diseases and life activities, and has become one of the important ways to analyze functions of noncoding RNA and protein.

It should be noted that the information disclosed in above section is only for enhancement of understanding of the background of the present disclosure, and therefore may contain information that does not form the prior art already known to a person of ordinary skill in the art.

### SUMMARY

The present disclosure provides a method and an apparatus for predicting an RNA-protein interaction, a computer-readable storage medium, and an electronic device.

The present disclosure provides a method for predicting an RNA-protein interaction, including: acquiring an RNA-protein pair to be predicted; obtaining a sequence feature of the RNA-protein pair to be predicted by performing feature extraction on the RNA-protein pair to be predicted; obtaining an RNA sequence representation vector and a protein sequence representation vector in the RNA-protein pair to be predicted by vectorizing the RNA-protein pair to be predicted; obtaining respectively by using multiple interaction prediction models, multiple interaction prediction values of the RNA-protein pair to be predicted, based on the sequence feature of the RNA-protein pair to be predicted, the RNA sequence representation vector and the protein sequence representation vector in the RNA-protein pair to be predicted; and determining an interaction between the RNA and the protein according to the multiple interaction prediction values.

In some exemplary embodiments of the present disclosure, said obtaining a sequence feature of the RNA-protein pair to be predicted by performing feature extraction on the RNA-protein pair to be predicted includes: obtaining an original sequence feature set; and determining a sequence feature of the RNA-protein pair to be predicted according to the original sequence feature set.

In some exemplary embodiments of the present disclosure, said determining a sequence feature of the RNA-protein pair to be predicted according to the original sequence feature set includes: converting an RNA sequence and a protein sequence in the RNA-protein pair to be predicted into k-mer subsequences, respectively; and searching for each of the k-mer subsequences in the original sequence feature set, and obtaining the sequence feature of the RNA-protein pair to be predicted according to a search result.

In some exemplary embodiments of the present disclosure, said determining a sequence feature of the RNA-protein pair to be predicted according to the original sequence feature set includes: converting an RNA sequence and a protein sequence in the RNA-protein pair to be predicted into k-mer subsequences, respectively, wherein the k-mer subsequences comprise an RNA k-mer subsequence and a protein k-mer subsequence; combining the RNA k-mer subsequence and the protein k-mer subsequence to obtain multiple RNA-protein k-mer subsequence pairs; and searching for each of the RNA-protein k-mer subsequence pairs in the original sequence feature set, and obtaining the sequence feature of the RNA-protein pair to be predicted according to a search result.

In some exemplary embodiments of the present disclosure, said determining a sequence feature of the RNA-protein pair to be predicted according to the original sequence feature set includes: converting an RNA sequence and a protein sequence in the RNA-protein pair to be predicted into k-mer subsequences, respectively, wherein the k-mer subsequences comprise an RNA k-mer subsequence and a protein k-mer subsequence; searching for each of the k-mer subsequences in the original sequence feature set to obtain a first sequence feature; combining the RNA k-mer subsequence and the protein k-mer subsequence to obtain multiple RNA-protein k-mer subsequence pairs; searching for each of the RNA-protein k-mer subsequence pairs in the original sequence feature set to obtain a second sequence feature; and forming the sequence feature of the RNA-protein pair to be predicted by using the first sequence feature and the second sequence feature.

In some exemplary embodiments of the present disclosure, said obtaining an original sequence feature set includes: obtaining an original data set; and performing feature extraction on each RNA-protein pair in the original data set to obtain the original sequence feature set.

In some exemplary embodiments of the present disclosure, said performing feature extraction on each RNA-protein pair in the original data set to obtain the original sequence feature set includes: obtaining k-mer subsequences by performing permutation with repetition on basic units of the RNA and the protein, respectively; counting frequency of occurrence of each of the k-mer subsequences in the original data set, and calculating variance of each of the k-mer subsequences according to the frequency of occurrence; and determining the original sequence feature set according to the variance of each of the k-mer subsequences.

In some exemplary embodiments of the present disclosure, said counting frequency of occurrence of each of the k-mer subsequences in the original data set, and calculating variance of each of the k-mer subsequences according to the frequency of occurrence includes: counting number of occurrence of each of the k-mer subsequences in the original data set; calculating the frequency of occurrence of each of the k-mer subsequences in the original data set according to the number of occurrence; marking whether each of the k-mer subsequences occurs in each RNA-protein pair by traversing the original data set; and calculating the variance of each of the k-mer subsequences according to the frequency of occurrence of each of the k-mer subsequences in the original data set and a marking value of each of the k-mer subsequences in each RNA-protein pair.

In some exemplary embodiments of the present disclosure, the calculation of each k-mer subsequence is based on the frequency of occurrence of each k-mer subsequence in the original data set and the marking value in each RNA-protein pair. The variance of k-mer subsequences, including: said calculating the variance of each of the k-mer subsequences according to the frequency of occurrence of each of the k-mer subsequences in the original data set and a marking value of each of the k-mer subsequences in each RNA-protein pair comprises calculating the variance Varᵢ of an i^{th} k-mer subsequence according to:

$${Var}_{i} = {\sum }_{n = 1}^{n = N} {( {Appear}_{{i}_{n}} - {Freq}_{i} )}^{2} ,$$

where Appearᵢ_{ₙ} is the marking value of the i^{th} k-mer subsequence in the n^{th} RNA-protein pair, Freqᵢ is the frequency of occurrence of the i^{th} k-mer subsequence in the original data set, and Nis a total number of RNA-protein pairs in the original data set.

In some exemplary embodiments of the present disclosure, said determining the original sequence feature set according to the variance of each of the k-mer subsequences includes: determining a k-mer subsequence that meets a preset condition according to the variance of each of the k-mer subsequences, and forming the original sequence feature set by using the k-mer subsequence that meets the preset condition.

In some exemplary embodiments of the present disclosure, said performing feature extraction on each RNA-protein pair in the original data set to obtain the original sequence feature set further includes: converting an RNA sequence and a protein sequence in each RNA-protein pair into k-mer subsequences, respectively, and forming a first candidate itemset by using the k-mer subsequences, wherein the k-mer subsequences comprise an RNA k-mer subsequence and a protein k-mer subsequence; counting frequency of occurrence of each of the k-mer subsequences contained in the first candidate itemset in the original data set, and forming a frequent itemset by using a k-mer subsequence that meets a preset occurrence frequency threshold; cross-combining the RNA k-mer subsequence and the protein k-mer subsequence contained in the frequent itemset, and forming a second candidate itemset by using a k-mer subsequence pair obtained through cross-combination; counting frequency of occurrence of each k-mer subsequence pair contained in the second candidate itemset in the original data set, to obtain a support degree of each k-mer subsequence pair; and forming the original sequence feature set by using a k-mer subsequence pair whose support degree meets a preset condition.

In some exemplary embodiments of the present disclosure, said obtaining an RNA sequence representation vector and a protein sequence representation vector in the RNA-protein pair to be predicted by vectorizing the RNA-protein pair to be predicted includes: converting an RNA sequence and a protein sequence in the RNA-protein pair to be predicted into k-mer subsequences, respectively, wherein the k-mer subsequences comprise M RNA k-mer subsequence and N protein k-mer subsequence; vectorizing each of the M RNA k-mer subsequences to obtain M RNA k-mer vectors; obtaining the RNA sequence representation vector by splicing the M RNA k-mer vectors; vectorizing each of the N protein k-mer subsequences to obtain N protein k-mer vectors; and obtaining the protein sequence representation vector by splicing the N protein k-mer vectors.

In some exemplary embodiments of the present disclosure, said obtaining respectively by using multiple interaction prediction models, multiple interaction prediction values of the RNA-protein pair to be predicted, based on the sequence feature of the RNA-protein pair to be predicted, the RNA sequence representation vector and the protein sequence representation vector in the RNA-protein pair to be predicted includes: inputting the sequence feature of the RNA-protein pair to be predicted into at least one first interaction prediction model to obtain at least one first interaction prediction value; and inputting the RNA sequence representation vector and the protein sequence representation vector in the RNA-protein pair to be predicted into at least one second interaction prediction model to obtain at least one second interaction prediction value.

In some exemplary embodiments of the present disclosure, said obtaining respectively by using multiple interaction prediction models, multiple interaction prediction values of the RNA-protein pair to be predicted, based on the sequence feature of the RNA-protein pair to be predicted, the RNA sequence representation vector and the protein sequence representation vector in the RNA-protein pair to be predicted includes: inputting the sequence feature of the RNA-protein pair to be predicted into at least one traditional machine learning model to obtain at least one first interaction prediction value; and inputting the RNA sequence representation vector and the protein sequence representation vector in the RNA-protein pair to be predicted into at least one deep learning model to obtain at least one second interaction prediction value.

In some exemplary embodiments of the present disclosure, each of the at least one deep learning model comprises at least two sub-deep learning models; and said inputting the RNA sequence representation vector and the protein sequence representation vector in the RNA-protein pair to be predicted into at least one deep learning model to obtain at least one second interaction prediction value includes: inputting the RNA sequence representation vector in the RNA-protein pair to be predicted into a first sub-deep learning model to obtain a first sequence feature; inputting the protein sequence representation vector in the RNA-protein pair to be predicted into a second sub-deep learning model to obtain a second sequence feature; and fusing the first sequence feature and the second sequence feature, and obtaining the second interaction prediction value according to a fused feature.

In some exemplary embodiments of the present disclosure, the traditional machine learning model comprises at least one of a logistic regression model, a support vector machine model and a decision tree model, and the deep learning model comprises at least one of a convolutional neural network model and a recurrent neural network model.

In some exemplary embodiments of the present disclosure, said determining an interaction between the RNA and the protein according to the multiple interaction prediction values includes: calculating a weighted sum of the multiple interaction prediction values; and determining the interaction between the RNA and the protein according to a calculation result.

In some exemplary embodiments of the present disclosure, said determining the interaction between the RNA and the protein according to a calculation result includes: determining the interaction between the RNA and the protein occurs in response to the calculation result being greater than a preset interaction prediction threshold; and determining the interaction between the RNA and the protein does not occur in response to the calculation result being less than or equal to the preset interaction prediction threshold.

In some exemplary embodiments of the present disclosure, the method further includes: training the multiple interaction prediction models jointly.

In some exemplary embodiments of the present disclosure, said training the multiple interaction prediction models jointly includes: obtaining a training data set, wherein the training data set comprises a positive RNA-protein pair and a negative RNA-protein pair; obtaining multiple interaction prediction values for each RNA-protein pair contained in the training data set by using the multiple interaction prediction models, respectively; obtaining a joint prediction value for each RNA-protein pair contained in the training data set according to the multiple interaction prediction values; performing calculation on the joint prediction value and a marking value for each RNA-protein pair contained in the training data set by using a loss function to obtain a corresponding loss value; and adjusting model parameters of the multiple interaction prediction models according to the loss value.

In some exemplary embodiments of the present disclosure, said obtaining a joint prediction value for each RNA-protein pair contained in the training data set according to the multiple interaction prediction values includes: obtaining the joint prediction value for each RNA-protein pair contained in the training data by calculating a weighted sum of the multiple interaction prediction values for each RNA-protein pair contained in the training data set.

In some exemplary embodiments of the present disclosure, said obtaining the joint prediction value for each RNA-protein pair contained in the training data by calculating a weighted sum of the multiple interaction prediction values for each RNA-protein pair contained in the training data set comprises calculating the joint prediction value yₒᵤₜ for each RNA-protein pair contained in the training data according to:

$${y}_{out} = α * {y}_{1} + β * {y}_{2} + γ * {y}_{3}$$

where y₁ is an output of the traditional machine learning model, y₂ is an output of the convolutional neural network model, y₃ is an output of the recurrent neural network model, and α, β, and γ are respectively weight parameters of the traditional machine learning model, the convolutional neural network model and the recurrent neural network model.

In some exemplary embodiments of the present disclosure, said adjusting model parameters of the multiple interaction prediction models according to the loss value includes: iteratively updating the model parameters of the multiple interaction prediction models based on the loss value, and ending training of the model parameters of the multiple interaction prediction models in response to satisfaction of an iteration termination condition, to predict the interaction of the RNA-protein pair to be predicted by using the multiple interaction prediction models trained.

In some exemplary embodiments of the present disclosure, the method further includes: outputting a prediction result of the interaction between the RNA and the protein.

The present disclosure provides apparatus for predicting an RNA-protein interaction, including: a data acquisition module configured to acquire an RNA-protein pair to be predicted; a feature extraction module configured to obtain a sequence feature of the RNA-protein pair to be predicted by performing feature extraction on the RNA-protein pair to be predicted; a data vectorization module configured to obtain an RNA sequence representation vector and a protein sequence representation vector in the RNA-protein pair to be predicted by vectorizing the RNA-protein pair to be predicted; an interaction prediction module configured to obtain respectively by using multiple interaction prediction models, multiple interaction prediction values of the RNA-protein pair to be predicted, based on the sequence feature of the RNA-protein pair to be predicted, the RNA sequence representation vector and the protein sequence representation vector in the RNA-protein pair to be predicted; and an interaction determination module configured to determine an interaction between the RNA and the protein according to the multiple interaction prediction values.

The present disclosure provides a computer-readable storage medium having a computer program stored thereon, wherein when the computer program is executed by a processor, cause any of the methods described above to be implemented.

The present disclosure provides an electronic device, including: a processor; and a memory for storing instructions executable by the processor; wherein the processor is configured to execute the instructions to cause any of the methods described above to be implemented.

It should be understood that the foregoing general description and the following detailed description are exemplary and explanatory only and do not limit the present disclosure.

### BRIEF DESCRIPTION OF THE DRAWINGS

The drawings, which are incorporated in and constitute a part of this specification, illustrate embodiments consistent with the present disclosure and serve together with the specification to explain the principle of the present disclosure. It is apparent that the drawings in the following description are only some embodiments of the present disclosure, and for those of ordinary skill in the art, other drawings can also be obtained from these drawings without creative efforts.

FIG. 1 shows a schematic diagram of an exemplary system architecture to which a method and an apparatus for predicting an RNA-protein interaction according to embodiments of the present disclosure can be applied;

FIG. 2 schematically shows a flow chart of a method for predicting an RNA-protein interaction according to embodiments of the present disclosure;

FIG. 3 schematically shows a process of determining a sequence characteristic of an RNA-protein pair to be predicted according to embodiments of the present disclosure;

FIG. 4 schematically shows a process of obtaining an original sequence feature set according to embodiments of the present disclosure;

FIG. 5 schematically shows a process of obtaining an original sequence feature set according to other embodiments of the present disclosure;

FIG. 6 schematically shows a process of training multiple interaction prediction models according to embodiments of the present disclosure;

FIG. 7 schematically shows a block diagram of an apparatus for predicting an RNA-protein interaction according to embodiments of the present disclosure; and

FIG. 8 shows a schematic structural diagram of a computer system suitable for implementing an electronic device according to embodiments of the present disclosure.

### DETAILED DESCRIPTION

Example embodiments will now be described more comprehensively with reference to the drawings. However, example embodiments can be embodied in various ways and should not be construed as limited to examples set forth herein. Instead, these embodiments are provided so that the present disclosure will be thorough and complete, and will fully convey the concept of example embodiments to those skilled in the art. The described features, structures, or characteristics may be combined in any suitable manner in one or more embodiments. In the following description, numerous specific details are provided in order to give a thorough understanding of embodiments of the present disclosure. However, those skilled in the art will appreciate that the technical solutions of the present disclosure may be practiced without one or more of the specific details, or other methods, components, devices, steps, etc., may be employed. In other instances, well-known solutions are not shown or described in detail to avoid obscuring aspects of the present disclosure.

Furthermore, the drawings are merely schematic illustrations of the present disclosure and are not necessarily drawn to scale. The same reference numerals in the drawings denote the same or similar parts, and thus their repeated descriptions will be omitted. Some of the block diagrams shown in the figures are functional entities that do not necessarily correspond to physically or logically separate entities. These functional entities may be implemented in software, or in one or more hardware modules or integrated circuits, or in different networks and/or processor devices and/or microcontroller devices.

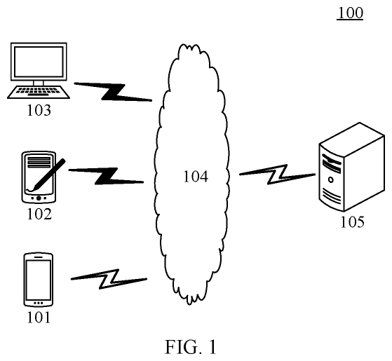

FIG. 1 shows a schematic diagram of a system architecture of an exemplary application environment to which a method and an apparatus for predicting an RNA-protein interaction according to embodiments of the present disclosure can be applied

As shown in FIG. 1, the system architecture 100 may include one or more terminal devices 101, 102, 103, a network 104 and a server 105. The network 104 is a medium used to provide a communication link between the terminal devices 101, 102, 103 and the server 105. The network 104 may include various connection types, such as wired, wireless communication links, or fiber optic cables, etc. The terminal devices 101, 102, and 103 may be various electronic devices, including but not limited to desktop computers, portable computers, smart phones, and tablet computers. It should be understood that the numbers of terminal devices, networks and servers in FIG. 1 are merely illustrative. There can be any number of terminal devices, networks and servers according to implementation needs. For example, the server 105 may be a server, a server cluster composed of multiple servers, or a cloud computing platform or a virtualization center. In some embodiments, the server 105 may be configured to: acquire an RNA-protein pair to be predicted; obtain a sequence feature of the RNA-protein pair to be predicted by performing feature extraction on the RNA-protein pair to be predicted; obtain an RNA sequence representation vector and a protein sequence representation vector in the RNA-protein pair to be predicted by vectorizing the RNA-protein pair to be predicted; obtain respectively by using multiple interaction prediction models, multiple interaction prediction values of the RNA-protein pair to be predicted, based on the sequence feature of the RNA-protein pair to be predicted, the RNA sequence representation vector and the protein sequence representation vector in the RNA-protein pair to be predicted; and determine an interaction between the RNA and the protein according to the multiple interaction prediction values.

The method for predicting the RNA-protein interaction provided by embodiments of the present disclosure is generally executed by the server 105. Accordingly, the apparatus for predicting the RNA-protein interaction is generally provided in the server 105. The server can send a prediction result of the interaction of RNA-protein pair to-be-predicted to the terminal device and displayed to the user by the terminal device. However, it is easily understood by those skilled in the art that the method for predicting the RNA-protein interaction provided by embodiments of the present disclosure can also be executed by one or more of the terminal devices 101, 102, and 103. Correspondingly, the apparatus for predicting the RNA-protein interaction can also be provided in the terminal devices 101, 102, 103. For example, after being executed by the terminal device, the prediction result can be directly displayed on a display screen of the terminal device, or the prediction result can be provided to the user by means of voice broadcast, which is not particularly limited in exemplary embodiments.

Technical solutions of embodiments of the present disclosure will be described in detail in the following.

Currently, noncoding RNA-protein interactions (ncRPI) can be studied using experimental methods. Traditional experimental methods can obtain valuable data experimentally to construct an ncRNA-protein interaction network, but are expensive and time-consuming.

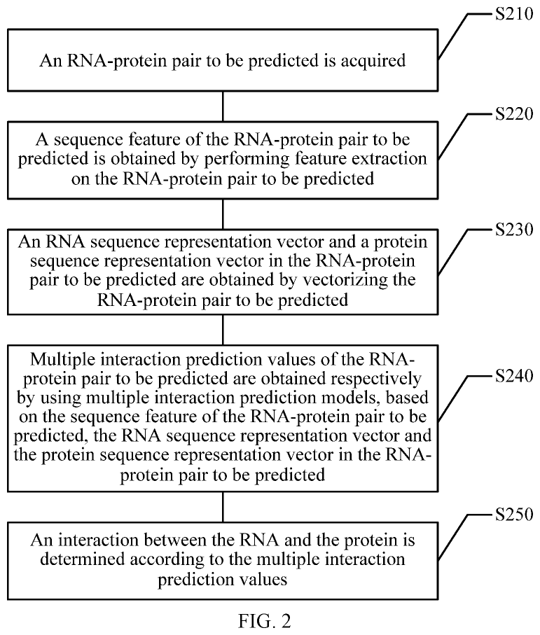

Exemplary embodiments of the present disclosure provide a method for predicting an RNA-protein interaction, which can be applied to above server 105, or one or more of above terminal devices 101, 102, and 103, which is not particularly limited in exemplary embodiments. Referring to FIG. 2, the method for predicting the RNA-protein interaction may include following steps S210 to S250.

In a step S210, an RNA-protein pair to be predicted is acquired.

In a step S220, a sequence feature of the RNA-protein pair to be predicted is obtained by performing feature extraction on the RNA-protein pair to be predicted.

In a step S230, an RNA sequence representation vector and a protein sequence representation vector in the RNA-protein pair to be predicted are obtained by vectorizing the RNA-protein pair to be predicted.

In a step S240, multiple interaction prediction values of the RNA-protein pair to be predicted are obtained respectively by using multiple interaction prediction models, based on the sequence feature of the RNA-protein pair to be predicted, the RNA sequence representation vector and the protein sequence representation vector in the RNA-protein pair to be predicted.

In a step S250, an interaction between the RNA and the protein is determined according to the multiple interaction prediction values.

In the method for predicting the RNA-protein interaction provided by exemplary embodiments of the present disclosure, an RNA-protein pair to be predicted is acquired; a sequence feature of the RNA-protein pair to be predicted is obtained by performing feature extraction on the RNA-protein pair to be predicted; an RNA sequence representation vector and a protein sequence representation vector in the RNA-protein pair to be predicted are obtained by vectorizing the RNA-protein pair to be predicted; multiple interaction prediction values of the RNA-protein pair to be predicted are obtained respectively by using multiple interaction prediction models, based on the sequence feature of the RNA-protein pair to be predicted, the RNA sequence representation vector and the protein sequence representation vector in the RNA-protein pair to be predicted; and an interaction between the RNA and the protein is determined according to the multiple interaction prediction values. According to embodiments, a relationship between an RNA sequence and a protein sequence can be fully mined through feature extraction and vectorization of the RNA-protein pair, so as to accurately predict the interaction between the RNA and the protein. According to embodiments, characteristics of multiple interaction prediction models can be combined effectively, which can further improve the accuracy of predicting the interaction between the RNA and the protein.

Hereinafter, the above steps in exemplary embodiments will be described in more detail.

In the step S210, the RNA-protein pair to be predicted is acquired.

In some example embodiments, at least one RNA-protein pair to be predicted can be acquired. The interaction between the RNA and the protein in each RNA-protein pair to be predicted is unknown. For example, a user can input the RNA-protein pair to be predicted through the terminal device. For example, the user can manually input the RNA-protein pair to be predicted, or can input the RNA-protein pair to be predicted through voice, which is not specifically limited in example embodiments. For example, an RNA can be input, and a protein can be input then, and an order of inputting the RNA and the protein is not limited. For example, the RNA and the protein can be entered into different text boxes or into the same text box. For example, after input of the RNA and the protein is completed, a “start prediction” button is clicked to start executing prediction steps provided in some embodiments of the present disclosure.

In some embodiments, the interaction between the RNA and the protein means that a function of the protein is reflected in the interaction between the protein and other proteins and RNA. For example, the interaction between the protein and the RNA plays an important role in protein synthesis. At the same time, performance of many functions of RNA is also inseparable from the interaction with proteins. The interaction can be regulation, guidance, etc., which is not limited herein. For example, in the presence of interaction, RNA can guide the synthesis of proteins, or RNA can regulate the function of protein. The interaction between the RNA and the protein can also refer to the two can regulate each other's life cycle and function through a physical interaction. For example, RNA coding sequences can guide the synthesis of proteins, and correspondingly, proteins can also regulate RNA expression and function.

After the RNA-protein pair to be predicted is acquired, multiple interaction prediction models can be used to predict the interaction of each input RNA-protein pair to be predicted, and whether there is an interaction between the RNA-protein pair to be predicted can be determined according to a prediction result. At the same time, the prediction result of the interaction of the RNA-protein pair to be predicted can also be output to the terminal device for the user to view. For example, the prediction result can be directly displayed on the display screen of the terminal device, or the prediction result may be provided to the user by means of voice broadcast, which is not specifically limited in exemplary embodiments.

In other examples, at least one RNA sequence to be predicted can be acquired, and a protein sequence that interacts with the input RNA sequence to be predicted can be searched in the database through multiple interaction prediction models. For example, after the RNA sequence to be predicted is inputted by the user through the terminal device, at least one protein sequence in the database can be selected, and the RNA sequence to be predicted and each of the at least one protein sequence can be combined to form multiple RNA-protein pairs. Then multiple interaction prediction models can be used to predict the interaction of each of the multiple RNA-protein pairs, and a protein sequence that can interact with the RNA sequence to be predicted can be output according to a prediction result. Preferably, several kinds of protein sequences can be pre-stored in the database for easy recall when predicting the interaction of the RNA-protein pair. For example, the protein sequence can be stored in the Redis database or in the MySQL database, and then the protein sequence to be predicted can be queried in real time and can be selected. Herein, Redis is a key-value storage system. Data stored in the Redis database includes a sequence identifier (such as a sequence number) and a key-value pair formed by a corresponding protein sequence. The key is the sequence identifier, and the value is the corresponding protein sequence. As an efficient caching technology, Redis can support a read and write frequency more than 100K+ per second, and has advantages in data reading and storage speed. MySQL is an associative database management system. The associative database stores data in different tables instead of storing all data uniformly, which increases the storage speed and improves the flexibility. MySQL has stable advantages in data storage and can avoid data loss.

It will be appreciated that several kinds of RNA sequences can also be pre-stored in the database for easy recall when predicting the interaction of RNA-protein pairs. Therefore, it is also possible to acquire at least one protein sequence to be predicted, and search the database for RNA sequences that interact with each of the at least one protein sequence to be predicted through multiple interaction prediction models. Similarly, after a protein sequence is inputted by the user through the terminal device, at least one RNA sequence in the database can be selected, and the protein sequence to be predicted and each of the at least one RNA sequence can be combined to form multiple RNA-protein pairs. Then multiple interaction prediction models can be used to predict the interaction of each of the multiple RNA-protein pairs, and an RNA sequence that can interact with the protein sequence to be predicted can be output according to a prediction result, which is not specifically limited in exemplary embodiments of the present disclosure.

In the step S220, the sequence feature of the RNA-protein pair to be predicted is obtained by performing feature extraction on the RNA-protein pair to be predicted.

In some exemplary embodiments, the description is taken where at least one RNA-protein pair to be predicted is acquired and the interaction of the at least one RNA-protein pair is predicted, as an example. Before predicting the interaction of each RNA-protein pair to be predicted through multiple interaction prediction models, an input feature of each interaction prediction model needs to be obtained. For example, feature extraction can be performed on the RNA-protein pair to be predicted, that is, feature extraction is performed on an RNA sequence and a protein sequence in the RNA-protein pair to be predicted, respectively, to obtain corresponding RNA sequence feature and protein sequence feature. The sequence feature of the RNA-protein pair to be predicted is composed of the RNA sequence feature and the protein sequence feature, and can be used as an input to the interaction prediction model. The RNA-protein pair to be predicted can also be vectorized, that is, the RNA sequence and the protein sequence in the RNA-protein pair are respectively vectorized to obtain corresponding RNA sequence representation vector and protein sequence representation vector, and the RNA sequence representation vector and the protein sequence representation vector are used as an input to the interaction prediction model, respectively. It is also possible to perform both feature extraction and vectorization processing on the RNA-protein pair to be predicted to obtain the sequence feature of the RNA-protein pair to be predicted, the RNA sequence representation vector and the protein sequence representation vector in the RNA-protein pair to be predicted. The sequence feature of the RNA-protein pair to be predicted, the RNA sequence representation vector and the protein sequence representation vector in the RNA-protein pair to be predicted can all be used as the input to the interaction prediction model, which are not specifically limited in exemplary embodiments of the present disclosure.

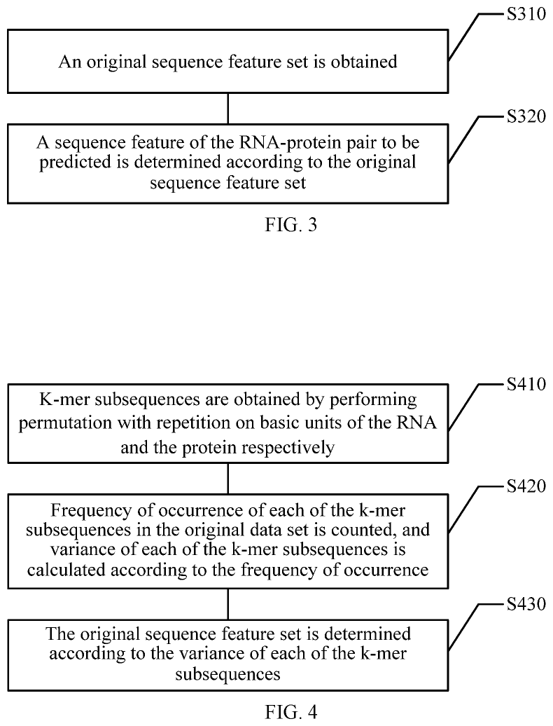

Referring to FIG. 3, the feature extraction can be performed on the RNA-protein pair to be predicted according to steps S310 and S320.

In a step S310, an original sequence feature set is obtained.

In some example embodiments, an original data set can be obtained, and the feature extraction is performed on each RNA-protein pair in the original data set to obtain the original sequence feature set. For example, the RPI1807 dataset can be used as the original data set, which contains 3243 RNA-protein pairs, and among the 3243 RNA-protein pairs, 1807 positive examples and 1436 negative examples are included. The positive examples can indicate that there is an interaction between the RNA and the protein in the RNA-protein pair, and the negative examples can indicate that there is no interaction between the RNA and the protein in the RNA-protein pair. It can be understood that in other examples, the RPI2241 dataset, the RPI369 dataset, etc., can also be used as the original data set for experiments, which are not specifically limited in exemplary embodiments of the present disclosure.

After the original data set is obtained, the feature extraction can be performed on the RNA-protein pairs in the original data set according to steps S410 to S430, referring to FIG. 4, to obtain the original sequence feature set.

In a step S410, k-mer subsequences are obtained by performing permutation with repetition on basic units of the RNA and the protein respectively.

For example, a base is taken as the basic unit of RNA. For the RNA sequence, four types of bases can be included, namely adenine (A), uracil (U), guanine (G) and cytosine (C). All k-mer subsequences of the RNA sequence can be obtained by performing permutation with repetition on the four types of bases. For example, the amino acid is taken as the basic unit of protein. For the protein sequence, 20 amino acids can be included, and the 20 amino acids can be encoded as A, G, V, I, L, F, P, Y, M, T, S, H, N, Q, W, R, K, D, E, and C. Exemplarily, 20 amino acids can be divided first, according to the physicochemical properties of amino acids, into {A, G, V}, {I, L, F, P}, {Y, M, T, S}, {H, N, Q, W}, {R, K}, {D, E} and {C}, a total of 7 types, and re-encode each type of amino acid. For example, the amino acids can be encoded as 1, 2, 3, 4, 5, 6 and 7. For example, the protein sequence ALQDVG (SEQ ID No. 1) can be converted to 124611. Then all the k-mer subsequences of the amino acid sequence can be obtained by performing permutation with repetition on the 7 types of amino acids. In other examples, the 20 amino acids can also be divided according to the amino acid composition, and the k-mer subsequences of the amino acid sequence can also be obtained by performing permutation with repetition directly on the 20 amino acids without classification, which is not specifically limited in exemplary embodiments of the present disclosure.

In exemplary embodiments of the present disclosure, the k-mer subsequence refers to a k-complex consisting of k bases or k-type amino acids as a group. Correspondingly, in exemplary embodiments of the present disclosure, the k-mer subsequences may include an RNA k-mer subsequence and a protein k-mer subsequence. Exemplarily, the k-mer subsequence may refer to an RNA k-mer subsequence obtained by performing permutation with repetition on 4 kinds of bases. For a certain k value, 4^{k} kinds of k-mer subsequences can be obtained. The k-mer subsequence can also refer to a protein k-mer subsequence obtained by performing permutation with repetition on 7 types of amino acids. For a certain k value, 7^{k} kinds of k-mer subsequences can be obtained. It can be understood that the classification of the 20 amino acids into 7 types is only illustrative, and the classification may not be performed. Similarly, the 4 bases of RNA sequences can also be classified according to actual needs.

In exemplary embodiments of the present disclosure, the value of k may take one or more values, and a specific value of k may be adjusted according to actual situations, which is not limited herein. In some examples, description is made by taking k takes two values of 3 and 4 as an example. There are 4³=64 kinds of 3-mer subsequences and 4⁴=256 kinds of 4-mer subsequences of RNA sequences in total. There are 7³=343 kinds of 3-mer subsequences and 7⁴=2401 kinds of 4-mer subsequences of protein sequences in total. For example, AAA and AUC are two 3-mer subsequences of the RNA sequence, and AAAA and AAAU are two 4-mer subsequences of the RNA sequence. 111 and 112 are two 3-mer subsequences of the protein sequence, and 1111 and 1122 are two 4-mer subsequences of the protein sequence. In other examples, k may also take only 3 or only 4, which is not specifically limited in exemplary embodiments of the present disclosure.

In a step S420, frequency of occurrence of each of the k-mer subsequences in the original data set is counted, and variance of each of the k-mer subsequences is calculated according to the frequency of occurrence.

In some exemplary embodiments, all 3-mer subsequences and 4-mer subsequences of the RNA sequence and the protein sequence can be obtained according to step S410. That is, 64 3-mer subsequences, 256 4-mer subsequences of the RNA sequence, and 343 3-mer subsequences, 2401 4-mer subsequences of the protein sequence are included. The frequency of occurrence of each 3-mer subsequence or 4-mer subsequence in the original data set can be counted, and the variance of each 3-mer subsequence or 4-mer subsequence can be calculated according to the frequency of occurrence. In some embodiments, before the frequency of occurrence of each 3-mer subsequence or 4-mer subsequence in the original data set is counted, it is necessary to convert the RNA sequence and the protein sequence of each RNA-protein pair in the original data set into 3-mer subsequences and 4-mer subsequences. For example, for the RNA sequence “AGAUGG”, the 3-mer subsequence of the sequence may include “AGA”, “GAU”, “AUG” and “UGG”, and the 4-mer subsequence of the sequence may include “AGAU”, “GAUG” and “AUGG”. That is, corresponding 3-mer subsequences or 4-mer subsequences can be obtained by reading the RNA sequence in a forward overlapping manner. Similarly, the corresponding 3-mer subsequences or 4-mer subsequences can also be obtained by reading the RNA sequence in a reverse overlapping manner. For example, the 3-mer subsequences of the sequence may also include “GGU”, “GUA”, “UAG” and “AGA”, and the 4-mer subsequences of the sequence may also include “GGUA”, “GUAG” and “UAGA”. In some embodiments, the RNA sequence can also be read in a nonoverlapping manner to obtain the corresponding 3-mer subsequences or 4-mer subsequences. For example, the 3-mer subsequences of the sequence can also include “AGA” and “UGG”, which is not specifically limited in exemplary embodiments of the present disclosure.

Exemplarily, number of occurrence of each 3-mer subsequence and/or 4-mer subsequence of the RNA sequence and the protein sequence in the original data set can be counted, and frequency of occurrence of each 3-mer subsequence and/or 4-mer subsequence of the RNA sequence and the protein sequence in the original data is calculated based on the number of occurrence. For example, the frequency of occurrence of a certain 3-mer subsequence in the original data set can be obtained by calculating a ratio between the number of occurrence of the certain 3-mer subsequence in the original data set and a total number of RNA-protein pairs in the original data set. Whether each 3-mer subsequence and/or 4-mer subsequence occurs in each RNA-protein pair is marked by traversing the original data set. The variance of each k-mer subsequence can be calculated from the frequency of occurrence of each 3-mer subsequence and/or 4-mer subsequence in the original data set and the marking value in each RNA-protein pair.

For example, for an i^{th} k-mer subsequence, the subsequence can be a 3-mer subsequence of the RNA sequence or a 3-mer subsequence of the protein sequence, or a 4-mer subsequence of the RNA sequence or 4-mer subsequence of the protein sequence. The number of occurrence of the subsequence in the RPI1807 dataset can be counted first. For example, N (N=3243) RNA-protein pairs in the RPI1807 dataset can be cycled, if the subsequence occurs in the current RNA-protein pair, the number of occurrence is incremented by 1. If the subsequence does not occur in the current RNA-protein pair, the number of occurrence remains unchanged, and then whether the subsequence occurs in next RNA-protein pair is determined. The number of occurrence of the i^{th} k-mer subsequence in the RPI1807 dataset is recorded as numᵢ. The frequency Freqᵢ of occurrence of the subsequence in the RPI1807 dataset can be calculated according to the number numᵢ of occurrence, which is

$${Freq}_{i} = \frac{{num}_{i}}{N} \qquad ( 1 )$$

After the frequency of occurrence of the i^{th} k-mer subsequence in the RPI1807 dataset is determined, the occurrence of this subsequence in each RNA-protein pair can be checked by traversing the RPI1807 dataset. The occurrence of this subsequence in each RNA-protein pair is marked, denoted as Appearᵢ_{ₙ}. That is, if the subsequence occurs in an n^{th} RNA-protein pair, the marking value Appearᵢ_{ₙ}=1, and if it does not occur in the n^{th} RNA-protein pair, the marking value Appearᵢ_{ₙ}=0.

After the frequency Freqᵢ of occurrence of the subsequence in the RPI1807 dataset and the marking value Appearᵢ_{ₙ} in the n^{th} RNA-protein pair are counted, the variance Varᵢ of the i^{th} k-mer subsequence in the RPI1807 dataset can be calculated:

$${Var}_{i} = {\sum }_{n = 1}^{n = N} {( {Appear}_{{i}_{n}} - {Freq}_{i} )}^{2} \qquad ( 2 )$$

A sum of the square of the difference between the marking value of the i^{th} k-mer subsequence in each RNA-protein pair in the RPI1807 dataset and the frequency of occurrence of the k-mer subsequence in the RPI1807 dataset is calculated to obtain the variance Varᵢ of the i^{th} k-mer subsequence in the RPI1807 dataset. Among which, Appear is the marking value of the i^{th} k-mer subsequence in the n^{th} RNA-protein pair, Freqᵢ is the frequency of occurrence of the i^{th} k-mer subsequence in the RPI1807 dataset, and N is total number of RNA-protein pairs in the RPI1807 dataset.

In a step S430, the original sequence feature set is determined according to the variance of each of the k-mer subsequences.

After the variance of each k-mer subsequence is calculated, the k-mer subsequence that meets a preset condition can be determined according to the variance of each k-mer subsequence, and the original sequence feature set is composed of the k-mer subsequence that meets the preset condition. Exemplarily, all 3-mer subsequences and 4-mer subsequences of the RNA sequence, and all 3-mer subsequences and 4-mer subsequences of the protein sequence can be sorted according to the variance size, for example, by a descending order. The top-ranked k-mer subsequences can be selected to form the original sequence feature set. For example, the top 560 k-mer subsequences can be selected, and the original sequence feature set can be composed of these 560 k-mer subsequences. Among which, it can include the top 60 3-mer subsequences of the RNA sequence, the top 200 4-mer subsequences of the RNA sequence, the top 200 3-mer subsequences of the protein sequence, and the top 100 4-mer subsequences of the protein sequence. It can be understood that the selected number of k-mer subsequences is only illustrative, and any number of k-mer subsequences can be selected according to actual requirements. In other examples, a variance threshold can also be preset, and k-mer subsequences with variance greater than the variance threshold are screened out, and the k-mer subsequences obtained by screening form the original sequence feature set. For example, when the preset variance threshold is 3, the k-mer subsequences with variance greater than 3 can be selected to form the original sequence feature set. It should be noted that when performing feature selection, a feature with a larger variance can be selected. The larger variance indicates that difference of the data having such feature is large. That is, the example can be better distinguished by using such feature, which can further improve the classification and prediction ability of interaction prediction models.

In some other exemplary embodiments, an average of the number of occurrence of each k-mer subsequence in each RNA-protein pair can also be calculated, and the variance of each k-mer subsequence can be calculated based on the average of the number of occurrence.

The original sequence feature set is then determined according to the variance of each k-mer subsequence.

In some embodiments, the number of occurrence of each 3-mer subsequence or 4-mer subsequence in each RNA-protein pair can be determined by traversing the original data set. The total number of occurrence of the subsequence in the original data set can be obtained by counting the number of occurrence of each 3-mer subsequence or 4-mer subsequence in each RNA-protein pair. The average of the number of occurrence of 3-mer subsequences or 4-mer subsequences in each RNA-protein pair can be obtained by calculating based on the total number of occurrence. The variance of each subsequence can be finally calculated based on the average of the number of occurrence of each 3-mer subsequence or 4-mer subsequence in each RNA-protein pair and the number of occurrence of each 3-mer subsequence or 4-mer subsequence in each RNA-protein pair.

For example, for the i^{th} k-mer subsequence, the subsequence can be a 3-mer subsequence of the RNA sequence or a 3-mer subsequence of the protein sequence, or a 4-mer subsequence of the RNA sequence or a 4-mer subsequence of the protein sequence. The total number of occurrence of this subsequence in the RPI1807 dataset can be counted first. For example, the number of occurrence of the subsequence in each RNA-protein pair can be counted by cycling n (n=3243) RNA-protein pairs in the RPI1807 dataset, and the number of occurrence can be denoted as x₁, x₂, . . . , xₙ. Then x₁, x₂, . . . , xₙ are superimposed to obtain the total number of occurrence of the subsequence in the RPI1807 dataset, denoted as numᵢ. The average mᵢ of the number of occurrence of the subsequence in each RNA-protein pair can be calculated according to the total number of occurrence numᵢ, based on:

$${m}_{i} = \frac{{num}_{i}}{n} \qquad ( 3 )$$

The average of the number of occurrence of the i^{th} k-mer subsequence in each RNA-protein pair is obtained through calculation. The variance of the i^{th} k-mer subsequence can be calculated from the average of the number of occurrence of the i^{th} k-mer subsequence in each RNA-protein pair and the number of occurrence of the i^{th} k-mer subsequence in each RNA-protein pair, that is, according to

$${s}^{2} = \frac{{( {m}_{i} - {x}_{1} )}^{2} + {( {m}_{i} - {x}_{2} )}^{2} + … + {( {m}_{i} - {x}_{n} )}^{2}}{n} \qquad ( 4 )$$

The variance s² of the i^{th} k-mer subsequence is calculated. Herein, n is the number of RNA-protein pairs in the RPI1807 dataset, mᵢ is the average of the number of occurrence of the subsequence in each RNA-protein pair, and xₙ is the number of occurrence of the subsequence in the n^{th} RNA-protein pair. Similarly, x₁ is the number of occurrence of the subsequence in the first RNA-protein pair, and x₂ is the number of occurrence of the subsequence in the second RNA-protein pair.

In some examples, after the variance of each k-mer subsequence is obtained through calculation, all k-mer subsequences can be sorted according to the variance, for example, in a descending order, and the top k-mer subsequences can be selected to form the original sequence feature set. In other examples, a variance threshold can also be preset, and k-mer subsequences with variance greater than the variance threshold are screened out, and the k-mer subsequences obtained by screening form the original sequence feature set.

In exemplary embodiments of the present disclosure, a k-mer feature of each RNA-protein pair in the original data set can be extracted, and the original sequence feature set is composed of the k-mer feature of the RNA sequence and the k-mer feature of the protein sequence extracted. The k-mer feature of the RNA sequence is taken as an example, the k-mer feature can contain monomer component information of the RNA sequence (i.e., each base contained) and sequence order information. Therefore, the RNA sequence can be better described by using the k-mer feature. That is, the RNA sequence can be more accurately determined according to the k-mer feature, and different RNA sequences can also be distinguished through the k-mer feature. In order to further mine the relationship between the RNA sequence and the protein sequence, a frequent itemset feature of each RNA-protein pair in the original data set can also be extracted, and the original sequence feature set is composed of the frequent itemset feature extracted. The frequent itemset feature can combine the k-mer feature of the RNA sequence with the k-mer feature of the protein sequence. Therefore, an interacting RNA-protein pair and a non-interacting RNA-protein pair can be better distinguished by using the frequent itemset feature. The k-mer feature and the frequent itemset feature can also be extracted at the same time, and the original sequence feature set can be formed by combining the two. The interaction between RNA and protein in unknown RNA-protein pairs can be predicted more accurately by combining the characteristics of the k-mer feature and the frequent itemset feature, which is not specifically limited in exemplary embodiments of the present disclosure. The frequent itemset feature refers to the k-mer subsequence pair composed of the RNA k-mer subsequence and the protein k-mer subsequence having a certain support degree in the original data set, and the support degree refers to a percentage of transactions that contain both A and B of all transactions. For example, the subsequence pair (AAU, 137) represents a 3-mer subsequence pair consisting of a 3-mer subsequence AAU of the RNA and a 3-mer subsequence 137 of the protein. The support degree of this subsequence pair is the ratio of RNA-protein pairs that contain both subsequences AAU and 137 to all RNA-protein pairs in the original data set.

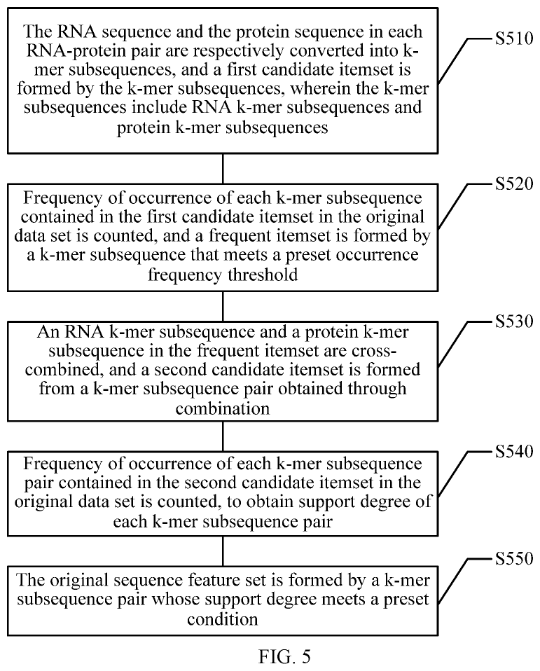

Referring to FIG. 5, the frequent itemset feature of each RNA-protein pair in the original data set can be extracted according to steps S510 to S550.

In a step S510, the RNA sequence and the protein sequence in each RNA-protein pair are respectively converted into k-mer subsequences, and a first candidate itemset is formed by the k-mer subsequences. The k-mer subsequences include RNA k-mer subsequences and protein k-mer subsequences.

In some exemplary embodiments, the RNA sequence and the protein sequence of each RNA-protein pair in the RPI1807 dataset can be first converted into 3-mer subsequences and 4-mer subsequences, respectively. All RNA 3-mer subsequences, RNA 4-mer subsequences, protein 3-mer subsequences and protein 4-mer subsequences in the dataset can be found by traversing the RPI1807 dataset, and all 3-mer subsequences and 4-mer subsequences in the dataset form the first candidate itemset C1.

In a step S520, frequency of occurrence of each k-mer subsequence contained in the first candidate itemset in the original data set is counted, and a frequent itemset is formed by a k-mer subsequence that meets a preset occurrence frequency threshold.

The frequency of occurrence of each k-mer subsequence contained in the first candidate itemset C1 in the original data set can be counted. Exemplarily, for a j^{th} k-mer subsequence, the subsequence may be a 3-mer subsequence of the RNA sequence or a 3-mer subsequence of the protein sequence, or a 4-mer subsequence of the RNA sequence or a 4-mer subsequence of the protein sequence. A number of occurrence of this subsequence in the RPI1807 dataset can be counted first. For example, N RNA-protein pairs in the RPI1807 dataset can be cycled, if the subsequence occurs in the current RNA-protein pair, the number of occurrence is increased by 1, and if the subsequence does not occur in the current RNA-protein pair, the number of occurrence remains unchanged. The number of occurrence of the j^{th} k-mer subsequence in the RPI1807 dataset is recorded as numᵢ. The frequency Freqⱼ of occurrence of the subsequence in the RPI1807 dataset can be calculated according to the number of occurrence numⱼ, which is:

$${Freq}_{j} = \frac{{num}_{j}}{N} \qquad ( 5 )$$

Similarly, the frequency of occurrence of each 3-mer subsequence or 4-mer subsequence contained in the first candidate itemset C1 in the RPI1807 dataset can be calculated. Then, all 3-mer subsequences and 4-mer subsequences can be screened according to the preset occurrence frequency threshold. For example, RNA 3-mer subsequences with an occurrence frequency greater than a first threshold, RNA 4-mer subsequences with an occurrence frequency greater than a second threshold, protein 3-mer subsequences with an occurrence frequency greater than a third threshold, and protein 4-mer subsequences with an occurrence frequency greater than a fourth threshold can be selected out together to form the frequent itemset L1. The first threshold, the second threshold, the third threshold and the fourth threshold may be the same or different, which are not specifically limited in embodiments of the present disclosure. In other examples, occurrence frequency of 3-mer subsequences and occurrence frequency of 4-mer subsequences of the RNA sequence, and occurrence frequency of 3-mer subsequences and occurrence frequency of 4-mer subsequences of the protein sequence can also be sorted in a descending order, and the frequent itemset L1 is formed by top ranked subsequences, which is not specifically limited in embodiments of the present disclosure.

In a step S530, an RNA k-mer subsequence and a protein k-mer subsequence in the frequent itemset are cross-combined, and a second candidate itemset is formed from a k-mer subsequence pair obtained through combination.

The RNA 3-mer subsequences and the protein 3-mer subsequences, the RNA 4-mer subsequences and the protein 4-mer subsequences in frequent itemset L1 can be cross-combined two by two respectively, to obtain a variety of 3-mer subsequence pairs and 4-mer subsequence pairs, and the second candidate itemset C2 is composed of multiple subsequence pairs obtained through combination. For example, if the frequent itemset includes [AAU, AUC, 137, 123, AAUU, AGUC, 1737, 1234], “AAU” and “137” can be combined to obtain a 3-mer subsequence pair “AAU_137”, or “AAU” and “123” can be combined to obtain a 3-mer subsequence pair “AAU_123”. Similarly, by cross-combining the RNA3-mer subsequence and the protein 3-mer subsequence, the 3-mer subsequence pairs “AUC_137” and “AUC_123” can also be obtained. By cross-combining the RNA 4-mer subsequence and the protein 4-mer subsequence, the 4-mer subsequence pairs “AAUU_1737”, “AAUU_1234”, “AGUC_1737” and “AGUC 1234” can be obtained.

In a step S540, frequency of occurrence of each k-mer subsequence pair contained in the second candidate itemset in the original data set is counted, to obtain support degree of each k-mer subsequence pair.

The frequency of occurrence of each subsequence pair contained in the second candidate itemset C2 in the RPI1807 dataset can be counted. Exemplarily, for an f^{th} k-mer subsequence pair, the subsequence pair may be a 3-mer subsequence pair or a 4-mer subsequence pair. A number of occurrence of this subsequence pair in the RPI1807 dataset can be counted first. For example, N RNA-protein pairs in the RPI1807 dataset can be cycled, if the subsequence pair occurs in the current RNA-protein pair, the number of occurrence is incremented by 1, and if the subsequence pair does not occur in the current RNA-protein pair, the number of occurrence remains unchanged. The number of occurrence of the f^{th} k-mer subsequence pair in the RPI1807 dataset is recorded as num_{f}, and the occurrence frequency of the subsequence pair in the RPI1807 dataset can be calculated according to the number of occurrence num_{f}, that is, the support degree support_{f} of the subsequence pair is obtained, which is:

$${support}_{f} = \frac{{num}_{f}}{N} \qquad ( 6 )$$

Similarly, the support degree of each subsequence pair in the second candidate itemset C2 can be calculated.

In a step S550, the original sequence feature set is formed by a k-mer subsequence pair whose support degree meets a preset condition.

After the support degree of each subsequence pair is calculated, the subsequence pair that meets the preset condition can be determined according to the support degree of each subsequence pair, and the original sequence feature set is formed by the subsequence pair that meets the preset condition. Exemplarily, a support degree threshold may be preset, then the subsequence pairs whose support degree are greater than the threshold are screened out, and the subsequence pairs screened form the original sequence feature set. For example, there are 370 subsequence pairs whose support degrees are greater than the threshold, these 370 subsequence pairs are 370 frequent itemset features, and the original sequence feature set can be formed by these 370 frequent itemset features. In other examples, all subsequence pairs in the second candidate itemset C2 can also be sorted in a descending order according to the support degree, and the top ranked subsequence pairs are selected to form the original sequence feature set, which is not specifically limited in embodiments of the present disclosure.

In some other exemplary embodiments, the RNA sequence and the protein sequence in each RNA-protein pair can be converted into k-mer subsequences, respectively, to obtain the k-mer subsequence pair. The frequency of occurrence of each k-mer subsequence pair in the original data set is counted, and the k-mer subsequence pair that meets the preset condition of occurrence frequency is used as the frequent itemset feature to form the original sequence feature set.

For example, the RNA sequence and the protein sequence of the RNA-protein pair of all positive examples in the RPI1807 dataset can be converted into positive 3-mer subsequence and positive 4-mer subsequence, respectively. Similarly, the RNA sequence and the protein sequence of the RNA-protein pair of all negative examples in the dataset are converted into negative 3-mer subsequence and negative 4-mer subsequence, respectively. All positive and negative RNA 3-mer subsequences, positive and negative RNA 4-mer subsequences, positive and negative protein 3-mer subsequences, and positive and negative protein 4-mer subsequences in the dataset can be found by traversing the RPI1807 dataset. The RNA 3-mer subsequence and the protein 3-mer subsequence, as well as the RNA 4-mer subsequence and the protein 4-mer subsequence in the dataset are cross-combined two by two, to obtain a variety of 3-mer subsequence pairs and 4-mer subsequence pairs. Exemplarily, the positive RNA 3-mer subsequence and the positive protein 3-mer subsequence can be cross-combined to obtain the positive 3-mer subsequence pair. The negative RNA3-mer subsequence and the negative protein 3-mer subsequence can be cross-combined to obtain the negative 3-mer subsequence pair. The positive RNA 4-mer subsequence and the positive protein 4-mer subsequence can be cross-combined to obtain the positive 4-mer subsequence pair. The negative RNA 4-mer subsequence and the negative protein 4-mer subsequence can be cross-combined to obtain the negative 4-mer subsequence pair.

The frequency of occurrence of each subsequence pair in the dataset can be counted. For example, for any positive 3-mer subsequence pair, the frequency Freq of occurrence of the positive 3-mer subsequence pair in the dataset can be calculated according to:

$$Freq = \frac{num}{NUM} \qquad ( 7 )$$

Herein, num is the number of occurrence of the positive 3-mer subsequence pair in the dataset, and NUM is the total number of occurrence of all positive 3-mer subsequence pairs in the dataset.

In some embodiments, after the frequency of occurrence of each k-mer subsequence pair in the original data set is calculated, all 3-mer subsequence pairs and 4-mer subsequence pairs can be sorted according to the occurrence frequency, such as sorted in a descending order, the top ranked k-mer subsequence pairs can be selected to form the frequent itemset. For example, all positive 3-mer subsequence pairs are sorted in a descending order, and the first m 3-mer subsequence pairs can be selected to form a frequent itemset A1. All positive 4-mer subsequence pairs are sorted in a descending order, and the first n 4-mer subsequence pairs can be selected to form a frequent itemset A2. All negative 3-mer subsequence pairs are sorted in a descending order, and the first p 3-mer subsequence pairs can be selected to form a frequent itemset A3. All negative 4-mer subsequence pairs are sorted in a descending order, and the first q 4-mer subsequence pairs can be selected to form a frequent itemset A4. The original sequence feature set is then formed by these four frequent itemsets A1, A2, A3 and A4. In other examples, an occurrence frequency threshold can also be preset, the k-mer subsequence pairs whose occurrence frequency are greater than the threshold are screened out, and the k-mer subsequence pairs obtained by screening are used as the frequent itemset features and form the original sequence feature set, which is not specifically limited in embodiments of the present disclosure.

In exemplary embodiments of the present disclosure, the frequent itemset feature can combine together the k-mer feature of the RNA sequence and the k-mer feature of the protein sequence, and an interacting RNA-protein pair and a non-interacting RNA-protein pair can be better distinguished by using the frequent itemset feature. Therefore, when the original sequence feature set is formed by frequent itemset features, and when the feature extraction of the RNA-protein pair to be predicted is performed based on the original sequence feature set, whether an interaction of the RNA-protein pair to be predicted occurs can be determined more accurately based on the extracted sequence features of the RNA-protein pair to be predicted.

In a step S320, a sequence feature of the RNA-protein pair to be predicted is determined according to the original sequence feature set.

In some exemplary embodiments, the RNA sequence and the protein sequence in the RNA-protein pair to be predicted can be converted respectively into k-mer subsequences. After the original sequence feature set is obtained, each k-mer subsequence can be searched in the original sequence feature set, and the sequence feature of the RNA-protein pair to be predicted is obtained according to a search result. The sequence feature of the RNA-protein pair to be predicted may refer to a complete sequence feature consisting of the RNA sequence feature and the protein sequence feature.

In some embodiments, the original sequence feature set may consist of 560 k-mer subsequences. For example, the 560 k-mer subsequences may be [CCC, . . . , AGU, CCCC, . . . CUGG, 777, . . . , 373, 7774, . . . , 7571]. The RNA-protein pair to be predicted is converted into 3-mer subsequences and 4-mer subsequences, to obtain RNA 3-mer subsequences, RNA 4-mer subsequences, protein 3-mer subsequences and protein 4-mer subsequences. The feature calculation can be performed on the RNA sequence and the protein sequence in the RNA-protein pair to be predicted based on the original sequence feature set, to obtain the sequence feature of the RNA-protein pair to be predicted

In some embodiments, for the original sequence feature set including 560 feature dimensions [CCC, . . . , AGU, CCCC, . . . , CUGG, 777, . . . , 373, 7774, . . . , 7571], a feature of each feature dimension corresponds to a kind of k-mer subsequence. For example, the subsequence CCC is the feature of the first feature dimension, and the subsequence 7571 is the feature of the 560^{th} feature dimension. All 3-mer subsequences and 4-mer subsequences of the RNA-protein pair to be predicted can be searched in the original sequence feature set, and whether a feature of each feature dimension in the original sequence feature set exists is determined according to a search result. If the feature of the feature dimension exists, a feature value for the feature dimension is 1, and if the feature of the feature dimension does not exist, a feature value for the feature dimension is 0. For example, if the RNA sequence in the RNA-protein pair to be predicted is AAAACCCGGG (SEQ ID No. 2), it can be seen that the feature CCC of the first feature dimension in the original sequence feature set is also a 3-mer subsequence of the RNA-protein pair. Therefore, it can be determined that the feature CCC of the first feature dimension in the original sequence feature set exists, and the feature value can be recorded as 1 correspondingly. For another example, the feature AGU in the original sequence feature set does not exist in the RNA sequence, therefore, the feature value for the feature dimension corresponding to the feature AGU in the original sequence feature set may be recorded as 0. Finally, a 560-dimensional feature value vector [1, 0, . . . , 0, 1, . . . ] can be obtained, and the feature value vector is the sequence feature of the RNA-protein pair to be predicted. It can be understood that each feature value contained in the feature value vector has a one-to-one correspondence with the feature value for each feature dimension in the original sequence feature set.

According to exemplary embodiments, the k-mer feature of each RNA-protein pair in the original data set is extracted, and the original sequence feature set is formed by the k-mer feature of the RNA sequence and the k-mer feature of the protein sequence extracted. The feature extraction is performed on the RNA-protein pair to be predicted based on the original sequence feature set, to obtain the sequence feature of the RNA-protein pair to be predicted. The k-mer feature of the RNA sequence is taken as an example, the k-mer feature may include the monomer composition information (i.e., each base contained) and the sequence order information. Therefore, the k-mer feature can be used to better describe an RNA sequence, that is, the RNA sequence can be more accurately determined according to the k-mer feature, and different RNA sequences can also be distinguished through the k-mer feature.

In some exemplary embodiments, after the original sequence feature set is obtained, the RNA sequence and the protein sequence in the RNA-protein pair to be predicted can be converted respectively into k-mer subsequences, and the RNA k-mer subsequence and the protein k-mer subsequence are cross-combined to obtain multiple RNA-protein k-mer subsequence pairs. Exemplarily, after the RNA 3-mer subsequence, the protein 3-mer subsequence, the RNA 4-mer subsequence and the protein 4-mer subsequence of the RNA-protein pair to be predicted are obtained, the RNA 3-mer subsequence and the protein 3-mer subsequence, as well as the RNA 4-mer subsequence and the protein 4-mer subsequence can be cross-combined two by two, to obtain a variety of 3-mer subsequence pairs and 4-mer subsequence pairs. Each RNA-protein k-mer subsequence pair can be searched for in the original sequence feature set, and the sequence feature of the RNA-protein pair can be obtained according to the search result.

Exemplarily, the original sequence feature set may be composed of 370 frequent itemset features. For example, the 370 frequent itemset features may be [CUG_122, AAU_122, . . . , CUUU_1312, UCUG 1312, . . . ]. The RNA-protein pair to be predicted is converted into 3-mer subsequences and 4-mer subsequences to obtain the RNA 3-mer subsequence, the RNA 4-mer subsequence, the protein 3-mer subsequence and the protein 4-mer subsequence. The RNA 3-mer subsequence and the protein 3-mer subsequence, as well as the RNA 4-mer subsequence and the protein 4-mer subsequence can be paired, to obtain various 3-mer subsequence pairs and 4-mer subsequence pairs. Then, feature calculation can be performed on the RNA sequence and the protein sequence in the RNA-protein pair to be predicted based on the original sequence feature set to obtain the sequence feature of the RNA-protein pair

In some embodiments, for the original sequence feature set including 370 feature dimensions [CUG 122, AAU_122, . . . , CUUU_1312, UCUG_1312, . . . ], the feature of each feature dimension corresponds to a k-mer subsequence pair. For example, the subsequence pair CUG 122 is the feature of the first feature dimension. All subsequence pairs of the RNA-protein pair to be predicted can be searched for in the original sequence feature set, and whether a feature of each feature dimension in the original sequence feature set exists is determined according to a search result. If the feature of the feature dimension exists, the feature value for the feature dimension is 1, and if the feature of the feature dimension does not exist, the feature value for the feature dimension is 0. For example, if the RNA sequence of the RNA-protein pair to be predicted is AUCUGAAAU, and the protein sequence is 512261312. It can be seen that CUG 122, AAU_122, and UCUG_1312 in the subsequence pair of the RNA-protein pair exist in the original sequence feature set. Therefore, the feature value for the corresponding feature dimension in the original sequence feature can be recorded as 1. Finally, a 370-dimensional feature value vector [1, 1, . . . , 0, 1, . . . ] can be obtained, and the feature value vector is the sequence feature of the RNA-protein pair to be predicted. Similarly, each feature value contained in the feature value vector has a one-to-one correspondence with the feature value for each feature dimension in the original sequence feature set.

In some other exemplary embodiments, after the original sequence feature set is obtained, the RNA sequence and the protein sequence in the RNA-protein pair to be predicted can be converted into k-mer subsequences respectively, and each k-mer subsequence can be searched for in the original sequence feature set, to obtain the first sequence feature. Then, the RNA k-mer subsequence and the protein k-mer subsequence can be combined to obtain a variety of RNA-protein k-mer subsequence pairs, and each RNA-protein k-mer subsequence pair is searched for in the original sequence feature set, to obtain the second sequence feature. Finally, the sequence feature of the RNA-protein pair to be predicted can be formed by the first sequence feature and the second sequence feature.

In some embodiments, the original sequence feature set may include two feature subsets. The two feature subsets respectively include 560 k-mer subsequences [CCC, . . . CCCC, . . . , 777, . . . , 7774, . . . ] and 370 frequent itemset features [CUG_122, AAU_122, . . . CUUU_1312, UCUG_1312, . . . ]. The RNA-protein pair to be predicted can be converted into RNA 3-mer subsequences, RNA 4-mer subsequences, protein 3-mer subsequences and protein 4-mer subsequences. Meanwhile, RNA 3-mer subsequences and protein 3-mer subsequences, as well as RNA 4-mer subsequences and protein 4-mer subsequences can be paired to obtain a variety of 3-mer subsequence pairs and 4-mer subsequence pairs. Then, feature calculation can be performed on the RNA sequence and the protein sequence in the RNA-protein pair to be predicted according to the original sequence feature set, so as to obtain the sequence feature of the RNA-protein pair to be predicted.

In some embodiments, all subsequences and subsequence pairs of the RNA-protein pair to be predicted can be searched for in the original sequence feature set, and whether a feature of each feature dimension in the original sequence feature set exists is determined according to a search result. For example, a 560-dimensional feature value vector [1, . . . , 1, . . . , 1, . . . , 0, . . . ] can be calculated and obtained by searching for all subsequences of the RNA-protein pair to be predicted, which is the first sequence feature. a 370-dimensional feature value vector [1, 1, . . . , 0, 1, . . . ] can be calculated and obtained by searching for all subsequence pairs of the RNA-protein pair, which is the second sequence feature. In some embodiments, a 930-dimensional feature value vector can be obtained by splicing these two feature value vectors, which is the sequence feature of the RNA-protein pair to be predicted. The two feature value vectors may also be directly input into the interaction prediction model at the same time, which is not specifically limited in embodiments of the present disclosure.

In other examples, a 930-dimensional original sequence feature set can also be formed by 560 k-mer subsequences and 370 frequent itemset features. For example, the original sequence feature set is [CCC, . . . , CCCC, . . . , 777, . . . , 7774, . . . , CCA_121, UCUG_1312, . . . , AAU_122, . . . , CUUU_1312, . . . ]. All subsequences and subsequence pairs of the RNA-protein pair can be searched for in the original sequence feature set, and whether the feature of each feature dimension in the original sequence feature set exists is determined according to a search result. A 930-dimensional feature value vector [1, . . . , 1, . . . , 1, . . . , 0, . . . , 1, . . . , 1, . . . , 1, . . . , 0, . . . ] is calculated and obtained, the feature value vector is the sequence feature of the RNA-protein pair to be predicted.

In order to further mine the relationship between RNA sequence and the protein sequence, the frequent itemset features of each RNA-protein pair in the original data set can also be extracted, and the original sequence feature set can be formed by the extracted frequent itemset features. The frequent itemset feature can combine the k-mer feature of the RNA sequence with the k-mer feature of the protein sequence. Therefore, an interacting and a non-interacting RNA-protein pairs can be better distinguished by using the frequent itemset feature. The k-mer feature and frequent itemset feature can also be extracted at the same time, and the original sequence feature set is formed by combining the two. By combining the characteristics of the k-mer feature and the frequent itemset feature, the interaction between the RNA and the protein in unknown RNA-protein pairs can be predicted more accurately.

In the step S230, the RNA sequence representation vector and the protein sequence representation vector in the RNA-protein pair to be predicted are obtained by vectorizing the RNA-protein pair to be predicted.

After feature extraction is performed on the RNA-protein pair to be predicted, the sequence feature obtained can be used as an input to a first interaction prediction model. In order to obtain sequence information of the RNA sequence and the protein sequence, as well as information between adjacent bases and amino acids, the RNA-protein pair to be predicted can also be vectorized, and an obtained vector can be used as an input to a second interaction prediction model.

In some exemplary embodiments, the RNA sequence and the protein sequence in the RNA-protein pair to be predicted can be converted to k-mer subsequences, respectively. For example, the RNA sequence can be divided into M RNA k-mer subsequences nonoverlappingly, and the protein sequence can be divided into N protein k-mer subsequences nonoverlappingly. For example, if the RNA sequence is AUCUGAAAU, it can be divided into three RNA k-mer subsequences, namely AUC, UGA and AAU. It can be understood that the RNA sequence and the protein sequence are divided into multiple k-mer subsequences nonoverlappingly, the purpose of which is to vectorize the RNA sequence and protein sequence, that is, the bases in the RNA sequence and the amino acids in the protein sequence are vectorized in the form of the k-complex. Similarly, in other examples, each base contained in the RNA sequence in the RNA-protein pair can also be vectorized to obtain multiple base vectors, and the multiple base vectors can be spliced to obtain the RNA sequence representation vector. At the same time, each amino acid contained in the protein sequence in the RNA-protein pair can be vectorized to obtain multiple amino acid vectors, and the protein sequence representation vector can be obtained by splicing the multiple amino acid vectors. The RNA sequence can also be divided into P RNA k-mer subsequences overlappingly, and the protein sequence can be divided into Q protein k-mer subsequences overlappingly, which are not specifically limited in embodiments of the present disclosure. The RNA sequence representation vector and the protein sequence representation vector can then be input into the second interaction prediction model, respectively.

Exemplarily, each k-mer subsequence of the RNA sequence and the protein sequence can be encoded first. Exemplarily, when k=3, there can be 64 RNA 3-mer subsequences and 343 protein 3-mer subsequences. Each RNA 3-mer subsequence and protein 3-mer subsequence can be encoded in turn by Embedding (vector mapping), and each 3-mer subsequence is represented by a low-dimensional vector, and corresponding multiple 3-mer subsequences can be obtained. In some examples, the One-Hot encoding can be performed on each 3-mer subsequence. The One-Hot encoding is also known as one-bit-efficient encoding. The One-Hot encoding method is to use an N-bit state register to perform encoding on N states, each state has independent register bit, and at any time, only one bit in the register is valid. For example, for an i^{th} RNA 3-mer subsequence, i.e., the RNA 3-mer subsequence whose index is an integer i, a 64-dimensional One-Hot vector can be obtained by encoding, and an i^{th} element in the vector is set to 1, all other elements are set to 0, in the form of [0, 1, 0, 0, . . . , 0]. For a j^{th} protein 3-mer subsequence, i.e., the protein 3-mer subsequence whose index is an integer j, a 343-dimensional One-Hot vector can be obtained by encoding, the a j^{th} element in the vector is set to 1, all other elements are set to 0. Similarly, each RNA 3-mer subsequence and protein 3-mer subsequence can correspond to a 3-mer One-Hot vector. In other examples, a dense vector can also be used to represent each 3-mer subsequence. For example, each 3-mer subsequence can be mapped into a vector space by using Word2vec algorithm, and each 3-mer subsequence can be represented by a subsequence vector in this vector space. Each 3-mer subsequence can also be encoded by using a BERT (Bidirectional Encoder Representations from Transformers) pretrained model, to obtain corresponding multiple 3-mer subsequence vectors. In some embodiments, large-scale RNA sequence data can be obtained, and the BERT pre-training model can be used for training. After the training is completed, a certain RNA sequence is inputted into the trained model, and a high-dimensional vector of the RNA sequence can be obtained, which is not specifically limited in embodiments of the present disclosure.

After all 3-mer One-Hot vectors of the RNA sequence and the protein sequence, the RNA sequence and the protein sequence in the RNA-protein pair to be predicted can be converted respectively into 3-mer subsequences. For example, M RNA 3-mer subsequences and N protein 3-mer subsequences can be obtained. The M 3-mer One-Hot vectors corresponding to the M RNA 3-mer subsequences can then be determined by querying, and the M 3-mer One-Hot vectors can be spliced in turn. For example, the splicing can be performed in a row direction to obtain an M*64 two-dimensional matrix, such as
[[0, 1, 0, 0, . . . ,]; [0, 0, 0, 1, . . . , 0]; . . . ; [1, 0, 0, 0, . . . , 0]]

The two-dimensional matrix is the 3-mer One-Hot representation vector of the RNA sequence. The N 3-mer One-Hot vectors corresponding to the N protein 3-mer subsequences can also be determined by querying, and the N 3-mer One-Hot vectors are spliced in sequence in the row direction to obtain an N*343 two-dimensional matrix. The two-dimensional matrix is the 3-mer One-Hot representation vector of the protein sequence. It can be understood that M 3-mer One-Hot vectors or N 3-mer One-Hot vectors can also be spliced in a column direction, and 3-mer One-Hot vectors of the sequence can also be obtained by direct (tail) splicing, which is not specifically limited in embodiments of the present disclosure.

In exemplary embodiments of the present disclosure, the RNA sequence and the protein sequence in the RNA-protein pair to be predicted are vectorized, and the RNA sequence representation vector and the protein sequence representation vector can be used as an input of a deep learning model, so as to further discover combinations of features that occur infrequently or are new in data, thereby revealing the interaction between implicit features.

In the step S240, multiple interaction prediction values of the RNA-protein pair to be predicted are obtained respectively by using multiple interaction prediction models, based on the sequence feature of the RNA-protein pair to be predicted, the RNA sequence representation vector and the protein sequence representation vector in the RNA-protein pair to be predicted.

In some example embodiments, the multiple interaction prediction models are obtained by jointly training. The multiple interaction prediction models may be all traditional machine learning models or all deep learning models, or may include at least one traditional machine learning model and at least one deep learning model at the same time. The traditional machine learning models refer to processing natural data in an original form. For example, constructing a pattern recognition or machine learning system requires extracting features from raw data (such as pixel values in an image) by using specialized knowledge, and converting the features into an appropriate feature representation. Exemplarily, traditional machine learning models may include linear regression models, logistic regression models, support vector machine models, decision tree models, K-Nearest Neighbor (KNN) models, random forest models, naive Bayesian models, etc. The deep learning model has the ability to automatically extract features, and can be composed of multiple processing layers to form a complex computing model, so as to automatically obtain data representation and multiple abstraction levels, which is a learning for feature representation. Exemplarily, the deep learning model may include a convolutional neural network model, a recurrent neural network model, and the like. When the multiple interaction prediction models are all traditional machine learning models, the RNA-protein pair to be predicted may not be vectorized, and only the sequence feature obtained by feature extraction of the RNA-protein pair to be predicted is used as the input to each traditional machine learning model. When the multiple interaction prediction models are all deep learning models, the feature extraction of the RNA-protein pair to be predicted may not be performed, and only the RNA sequence representation vector and the protein sequence representation vector obtained by vectorizing the RNA-protein pair to be predicted are used as the input to various deep learning models.

After the sequence feature of the RNA-protein pair to be predicted, the RNA sequence representation vector and the protein sequence representation vector in the RNA-protein pair to be predicted are obtained, the sequence feature of the RNA-protein pair can be input into at least one first interaction prediction model, to obtain at least one first interaction prediction value. Meanwhile, the RNA sequence representation vector and the protein sequence representation vector in the RNA-protein pair to be predicted can be input into at least one second interaction prediction model to obtain at least one second interaction prediction value. It should be noted that the first interaction prediction model and the at least one second interaction prediction model are obtained based on joint learning. The joint learning refers to combining multiple sub-models into one model to complete the final target task. Similarly, in exemplary embodiments of the present disclosure, at least one first interaction prediction model and at least one second interaction prediction model can be combined, and a final prediction result can be obtained by fusing the outputs of respective models. Moreover, in a model training process, respective models and a weighted sum result can be considered at the same time, and the model parameters of each model can be optimized at the same time, to obtain a best overall model, thereby improving the prediction ability of the overall model.

In some example implementations, the first interaction model may be a traditional machine learning model, and the second interaction model may be a deep learning model. Correspondingly, the sequence feature of the RNA-protein pair to be predicted can be input into at least one traditional machine learning model to obtain at least one first interaction prediction value. The RNA sequence representation vector and the protein sequence representation vector in the RNA-protein pair to be predicted can be input into at least one deep learning model to obtain at least one second interaction prediction value. It should be noted that each deep learning model may include at least two sub-deep learning models, which are respectively used to process the RNA sequence representation vector and the protein sequence representation vector in the RNA-protein pair to be predicted. For example, the RNA sequence representation vector can be input into a first sub-deep learning model to obtain a first sequence feature. At the same time, the protein sequence representation vector can be input into a second sub-deep learning model to obtain a second sequence feature. Finally, the first sequence feature and the second sequence feature can be fused through the fully connected layer of each deep learning model, and the second interaction prediction value can be obtained according to the fused feature.

In some exemplary embodiments of the present disclosure, the traditional machine learning model may include at least one of an LR (Logistic Regression) model, an SVM (Support Vector Machine) model, and a decision tree model. The deep learning model may include at least one of a CNN (Convolutional Neural Network) model and a recurrent neural network model. The recurrent neural network model may be an LSTM (Long Short-Term Memory) model, a BiLSTM (Bi-directional LSTM) model.

Description is made by taking an example where the joint learning of the LR model, the CNN model and the BiLSTM model is performed to predict the interaction of RNA-protein pairs. Exemplarily, a 930-dimensional original sequence feature set can be composed of 560 k-mer subsequences and 370 frequent itemset features. The feature calculation is performed on the original sequence feature set according to the k-mer subsequence pair of the RNA-protein pair to be predicted, to obtain a 930-dimensional feature value vector. The feature value vector is used as the input to the LR model to obtain an interaction prediction value y₁. At the same time, the RNA-protein pair to be predicted can also be vectorized to obtain the k-mer One-Hot vectors of the RNA sequence and the protein sequence, respectively, which can be used as the input to the CNN model and the BiLSTM model, respectively, to obtain the interaction prediction values y₂, y₃.

In some exemplary embodiments of the present disclosure, the LR model has good memory ability, and can learn the correlation between sequences or features from data. The CNN model and the BiLSTM model have good generalization ability, and can discover combinations of features that occur infrequently or are new in data, and reveal the interaction between implicit features. The CNN model can better capture the features but ignore the location information of the features, while the BiLSTM model has better memory ability and can use the sequence information and location information of the data to make up for defects of the CNN model in memory ability. The LR model is simple and has good interpretability. The joint learning of the CNN model, the BiLSTM model and the LR model can enhance the interpretability of the RPI prediction by the overall model. In general, the characteristics of each interaction prediction model can be effectively combined by the present disclosure, so that the prediction ability of the overall model can be improved.

In the step S250, the interaction between the RNA and the protein is determined according to the multiple interaction prediction values.

After the multiple interaction prediction values are obtained, a weighted sum is calculated based on the multiple interaction prediction values, and the interaction between the RNA and the protein can be determined according to a calculation result. If the calculated result is greater than a preset interaction prediction threshold, it can be determined that there is an interaction between the RNA and the protein. If the calculated result is less than or equal to the preset interaction prediction threshold, it can be determined that there is no interaction between the RNA and the protein. A final prediction result can be obtained by fusing the output values of each interaction prediction model, so that the interaction between the RNA and the protein in the RNA-protein pair to be predicted can be determined more accurately.

Exemplarily, the interaction of RNA-protein pairs can be predicted according to the following formula:

$${y}_{out} = α * {y}_{1} + β * {y}_{2} + γ * {y}_{3}$$

The interaction prediction value yₒᵤₜ of the RNA-protein pair to be predicted is calculated and obtained. Among which, y₁ is an output of the logistic regression model, y₂ is an output of the convolutional neural network model, y₃ is an output of the recurrent neural network model, and α, β, and γ are respectively weight parameters of the logistic regression model, the convolutional neural network model and the recurrent neural network model. The interaction prediction value yₒᵤₜ can be any value between 0-1. Exemplarily, the interaction prediction threshold can be preset as 0.5. When yₒᵤₜ>0.5, the prediction result can be marked as 1, which means that an interaction of the RNA-protein pair occurs. When yₒᵤₜ<0.5, the prediction result can be marked as 0, which means that the interaction of the RNA-protein pair has not occurred.

For example, above weight parameters α, β, and γ can be obtained based on jointly learning training.

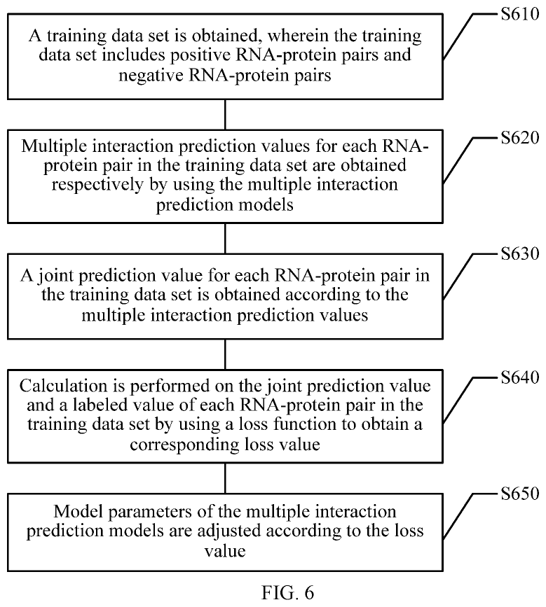

In some exemplary embodiments of the present disclosure, referring to FIG. 6, the multiple interaction prediction models and related parameters can be jointly trained in advance according to steps S610 to S650, to realize optimization of all model parameters in each prediction model. The resulting final model obtained by training can be used to make predictions for RNA-protein pairs with unknown interactions.

In a step S610, a training data set is obtained. The training data set includes positive RNA-protein pairs and negative RNA-protein pairs.

All RNA-protein pairs in the original data set can be used as the training data set, or part of the RNA-protein pairs in the original data set can be used as the training data set. The RPI1807 dataset is taken as an example, there are a total of 3243 RNA-protein pairs in this dataset, including 1807 positive examples and 1436 negative examples. Exemplarily, 1200 positive examples and 1000 negative examples can be selected as the training data set. It can be understood that the number of RNA-protein pairs in the training dataset is only illustrative, and any number of RNA-protein pairs can be obtained to train each interaction prediction model for multiple times to improve the performance of each interaction prediction model. performance. It should be noted that the positive RNA-protein pair can be labeled, and an obtained labeled value is “1”, which means that the interaction of the RNA-protein pair occurs. The negative RNA-protein pair can be labeled, and an obtained labeled value is “0”, which means that the interaction of the RNA-protein pair has not occurred.

In a step S620, multiple interaction prediction values for each RNA-protein pair in the training data set are obtained respectively by using the multiple interaction prediction models.

For example, the multiple interaction prediction models can be the LR model, the CNN model and the BiLSTM model respectively. Similarly, feature extraction is performed on each RNA-protein pair in the training data set, and the extracted sequence feature can be sequentially input into the LR model. Vectorization is performed on each RNA-protein pair, and the resulting RNA sequence representation vector and the protein sequence representation vector can be input into the CNN model and the BiLSTM model. Exemplarily, the training of each prediction model using the i^{th} RNA-protein pair is taken as an example. The sequence feature of the RNA-protein pair can be input into the LR model and an output predicted value y₁ is obtained. The RNA sequence representation vector and the protein sequence representation vector in the RNA-protein pair can be input into two CNN sub-models respectively, and outputs of the two CNN sub-models can be spliced to finally obtain an output predicted value y₂. Similarly, the RNA sequence representation vector and the protein sequence representation vector in the RNA-protein pair can be input into the BiLSTM model, and an output predicted value y₃ can be obtained. It can be understood that, for each RNA-protein pair in the training data set, three interaction prediction values can be obtained respectively through the three interaction prediction models.

In a step S630, a joint prediction value for each RNA-protein pair in the training data set is obtained according to the multiple interaction prediction values.

After the multiple interaction prediction values for each RNA-protein pair in the training data set are obtained, the multiple interaction prediction values can be weighted and summed to obtain the joint prediction value for each RNA-protein pair. Still the i^{th} RNA-protein pair in the training data set is taken as an example, the joint prediction value yₒᵤₜ of the RNA-protein pair can be determined according to:

$${y}_{out} = α * {y}_{1} + β * {y}_{2} + γ * {y}_{3} ,$$

where y₁ is the output of the LR model, y₂ is the output of the CNN model, y₃ is the output of the BiLSTM model, and α, β, and γ are respectively weight parameters of the LR model, the CNN model and the BiLSTM model.

In a step S640, calculation is performed on the joint prediction value and a labeled value of each RNA-protein pair in the training data set by using a loss function to obtain a corresponding loss value.

For each RNA-protein pair in the training data set, there is a labeled value. For example, the labeled value of each positive example pair is 1, and the labeled value of each negative example pair is 0. Exemplarily, the i^{th} RNA-protein pair is a positive example, and its corresponding labeled value is 1. The loss function can be calculated according to the joint prediction value yₒᵤₜ and the labeled value 1 of the RNA-protein pair, to obtain the corresponding loss value. In the training process of the prediction model, it is necessary to make the joint prediction value infinitely close to the labeled value, that is, to minimize an objective function. In some examples, when the objective function needs to be minimized, the cross-entropy loss function can be selected as the objective function. When the cross-entropy loss function is calculated, if the labeled value is 1, the closer yₒᵤₜ gets to 1, the smaller the calculated loss value is, and the closer yₒᵤₜ gets to 0, the larger the calculated loss value is. If the labeled value is 0, the closer yₒᵤₜ gets to 0, the smaller the calculated loss value is, and the closer yₒᵤₜ gets to 1, the larger the calculated loss value is. It can be understood that the cross-entropy loss function is a performance function in the prediction model and can be used to measure inconsistency between the predicted value from the prediction model and the labeled value. The smaller the value of the calculated cross-entropy loss function, the better effect the prediction model has.

In a step S650, model parameters of the multiple interaction prediction models are adjusted according to the loss value.

The model parameters of the multiple interaction prediction models can be iteratively updated based on the calculated loss values, and when an iteration termination condition is satisfied, the training of the model parameters and the weight parameters of the multiple interaction prediction models is completed. Exemplarily, the parameters can be updated by using a stochastic gradient descent algorithm. According to the principle of back propagation, the objective function such as the cross-entropy loss function is continuously calculated, and the model parameters and weight parameters of each interaction prediction model are simultaneously updated according to the calculated loss value. When the objective function converges to the minimum value, the training of all model parameters is completed. The model parameters can also be updated iteratively in the reverse direction, and when the preset number of iterations is satisfied, the training of all model parameters is completed. Exemplarily, the preset number of iterations may be 20, and each interaction prediction model is constantly updating model parameters during the 20 reverse iterations. After the iteration is completed, the optimized model parameters can be obtained. In other examples, the objective function can also be minimized by alternating least squares method, Adam optimization algorithm, etc., and the model parameters and the weight parameters can be updated sequentially from back to front to optimize the parameters. It should be noted that the weight parameters α, β, and γ are also part of the model parameters, and the weight parameters will also be trained and continuously optimized in the process of joint training of the multiple interaction prediction models.

In some exemplary embodiments of the present disclosure, by using the multiple interaction prediction models to predict the interaction of unknown RNA-protein pairs, and by combining the logistic regression model with the deep learning model, the logistic regression model is used to learn correlation between each k-mer feature and/or each frequent itemset feature from the original data set, and the deep learning model is used to reveal the interaction between implicit features, so that the characteristics of the CNN model and the BiLSTM model can be combined, and at the same time the model parameters of each model are optimized in the process of joint training of multiple models, thereby improving the accuracy of predicting the interaction of unknown RNA-protein pairs by the overall model.

In some other example implementations, when the multiple interaction prediction models are jointly trained in advance, the original data set can be divided into a training data set, a validation data set and a test data set in proportion. Exemplarily, the RPI1807 dataset is taken as the original data set an example. There are a total of 3243 RNA-protein pairs in this dataset, including 1807 positive examples and 1436 negative examples. Exemplarily, the dataset can be divided into a training data set, a validation data set and a test data set according to a ratio of 7:2:1. In some embodiments, a ratio of positive and negative examples in each data set can be consistent with the distribution of the overall dataset, that is, the ratio is 1807:1436, which is about 1.25:1. For example, 1250 positive examples and 1000 negative examples can be selected as the training data set, 360 positive examples and 280 negative examples can be selected as the validation data set, and 180 positive examples and 140 negative examples can be selected as the test data set

The training data set can be input into multiple interaction prediction models, and the corresponding model parameters can be determined by using the back-propagation algorithm to obtain a first joint model. Exemplarily, the training data set can be input into each interaction prediction model, and the model parameters can be adjusted. For example, the model parameters can be weight parameters, bias parameters, intercept parameters, etc. Exemplarily, each model parameter can be updated by using a stochastic gradient descent algorithm. According to the principle of back propagation, the objective function is continuously calculated, and the model parameters are updated according to the objective function. When the objective function converges to the minimum value, the training of the model parameters is completed, thereby obtaining the first joint model.

The verification data set can be input into the first joint model to verify the performance of the first joint model, and a second joint model can be obtained according to a verification result. Exemplarily, when optimization is performed on the model parameters, a set of hyperparameters may be initialized first, and the multiple interaction prediction models may be continuously trained by using the training data set to obtain the first joint model. The hyperparameters can be a learning rate, number of CNN layers, a size of a convolution kernel, etc. Then, the validation data set can be input into the trained first joint model to verify the prediction accuracy of the first joint model. When the prediction accuracy reaches a preset accuracy threshold, the current first joint model can be used as the second joint model, that is, the final trained model is obtained. Finally, the final performance of the model can be tested by using the test data set on this trained model. It can be understood that if the prediction effect of the second joint model is poor according to the test data set, a set of hyperparameters can be reset, and the interaction prediction model can be trained and tested again by using the training data set and the verification data set. When the prediction accuracy for the validation data set obtained by the trained interaction prediction model reaches a preset accuracy threshold, the final performance of the prediction model can be tested by using a new test data set.

Exemplarily, the accuracy rate of the second joint model may be determined according to the test data set, and when the accuracy rate is greater than a preset threshold, a third joint model is obtained. The third joint model includes multiple trained interaction prediction models. For example, after the second joint model is obtained, each RNA-protein pair in the test data set can be input into the second joint model to determine the accuracy of the second joint model. If the accuracy of the model is greater than a preset accuracy threshold, the third joint model is obtained. It can be understood that the third joint model includes multiple trained interaction prediction models, and then the multiple interaction prediction models can be used to predict the interaction of unknown RNA-protein pairs. The test data set can also be used to determine the Matthews correlation coefficient of the second joint model. The Matthews correlation coefficient refers to a correlation coefficient between an actual classification and a predicted classification, and its value range is [0, 1]. A larger value indicates that the predicted value is more related to the actual value, and a value of 1 indicates that the predicted result is completely correct. If the Matthews correlation coefficient of the model is greater than a preset threshold, the third joint model is obtained. In some embodiments, a specificity rate, a recall rate, etc., of the second joint model may also be judged by using the test data set, which is not specifically limited in embodiments of the present disclosure. For example, if the accuracy of the second joint model is not greater than the preset accuracy threshold, a new training data set may be obtained to train the model parameters of each interaction prediction model again, so as to continuously improve the performance of the model. In exemplary embodiments of the present disclosure, the parameters can be adjusted by using the training data set and the validation data set to obtain the best prediction model by training, and then the generalization performance of the prediction model can be tested by using the test data set

In above training process, the model parameters of the LR model, the CNN model and the BiLSTM model can be trained at the same time. Exemplarily, based on the objective function, the model parameters of the three interaction prediction models can be adjusted at the same time according to the loss value calculated from the joint prediction value and the labeled value of each RNA-protein pair in the training data set, and through multiple back propagation, the model parameters of the three interaction prediction models can finally all tend to converge, or the training can be terminated after a certain number of iterations. Through such training method, the three interaction prediction models of the LR model, the CNN model and the BiLSTM model can be trained at the same time, thereby realizing the joint learning of the three interaction prediction models. At the same time, it can not only ensure higher precision and accuracy of each interaction prediction model in predicting RNA-protein pair interactions, but also improve the training efficiency of each interaction prediction model.

After the training of each model is completed, the interaction of the unknown RNA-protein pair can be predicted by using each interaction prediction model finally obtained, and the prediction result of the interaction of the RNA-protein pair can be output to the terminal device for the user to view.

In some example implementations, the same training dataset, validation dataset, and test dataset may be used to train and test separate LR model, CNN model, BiLSTM model, and the joint learning model for the three. For example, the performance of each prediction model can be evaluated by using two performance indicators, namely the accuracy rate and Matthews correlation coefficient. When the RPI1807 dataset is used for model training and testing, the accuracy rate and Matthews correlation coefficient of the joint learning model are better than those of the LR model, the CNN model, and the BiLSTM model. That is, the joint learning model has better performance than each single model. That is, the joint learning model has better prediction ability.

In exemplary embodiments of the present disclosure, at least one RNA sequence can also be obtained, and a protein sequence that interacts with each input RNA sequence can be searched for in the database through multiple interaction prediction models. In some embodiments, after the original data set is obtained, the multiple interaction prediction models can be jointly trained in advance by using the original data set with reference to FIG. 6. After the training is completed, all protein sequences participating in the joint training can be stored in the database. It should be noted that the database can also include other protein sequences that do not participate in joint training, that is, the number and the type of protein sequences in the database can be arbitrary. The database can also include any number of RNA sequences. For example, the RNA sequence includes but is not limited to all RNA sequences participating in joint training, which is not specifically limited in embodiments of the present disclosure.

After at least one RNA sequence is input by the user, each input RNA sequence can be combined with all protein sequences in the database into several RNA-protein pairs. Further, jointly learning can be conducted by the multiple interaction models according to steps S220 to S250, to predict the interaction of each RNA-protein pair. In some embodiments, feature extraction and vectorization can be performed on each RNA-protein pair, and the obtained sequence features, the RNA sequence representation vector and the protein sequence representation vector in the RNA-protein pair are input into multiple interaction prediction models, and predicted interaction values are obtained for each RNA-protein pair. An interaction prediction value of 1 indicates that the interaction of the RNA-protein pair occurs, and an interaction prediction value of 0 indicates that the interaction of the RNA-protein pair has not occurred. Then, all RNA-protein pairs with an interaction prediction value of 1 can be screened out, and the protein sequence in each RNA-protein pair can be output to the terminal device for the user to view the protein sequence that interacts with the input RNA sequence.

Similarly, in exemplary embodiments of the present disclosure, at least one protein sequence can also be obtained, and an RNA sequence that interacts with each input protein sequence can be searched for in the database through multiple interaction prediction models. Exemplarily, after at least one protein sequence is input by the user, each input protein sequence can be combined with all RNA sequences in the database to form several RNA-protein pairs. Further, jointly learning can be conducted by the multiple interaction models according to steps S220 to S250, to predict the interaction of each RNA-protein pair. In some embodiments, feature extraction and vectorization can be performed on each RNA-protein pair, and the obtained sequence features, the RNA sequence representation vector and the protein sequence representation vector in the RNA-protein pair are input into multiple interaction prediction models, and predicted interaction values are obtained for each RNA-protein pair. An interaction prediction value of 1 indicates that the interaction of the RNA-protein pair occurs, and an interaction prediction value of 0 indicates that the interaction of the RNA-protein pair has not occurred. Then, all RNA-protein pairs with an interaction prediction value of 1 can be screened out, and the RNA sequence in each RNA-protein pair can be output to the terminal device for the user to view the RNA sequence that interacts with the input protein sequence.

In the method for predicting the RNA-protein interaction provided by exemplary embodiments of the present disclosure, an RNA-protein pair to be predicted is acquired; a sequence feature of the RNA-protein pair to be predicted is obtained by performing feature extraction on the RNA-protein pair to be predicted; an RNA sequence representation vector and a protein sequence representation vector in the RNA-protein pair to be predicted are obtained by vectorizing the RNA-protein pair to be predicted; multiple interaction prediction values of the RNA-protein pair to be predicted are obtained respectively by using multiple interaction prediction models, based on the sequence feature of the RNA-protein pair to be predicted, the RNA sequence representation vector and the protein sequence representation vector in the RNA-protein pair to be predicted; and an interaction between the RNA and the protein is determined according to the multiple interaction prediction values. According to embodiments, a relationship between an RNA sequence and a protein sequence can be fully mined through feature extraction and vectorization of the RNA-protein pair, so as to accurately predict the interaction between the RNA and the protein. According to embodiments, characteristics of multiple interaction prediction models can be combined effectively, which can further improve the accuracy of predicting the interaction between the RNA and the protein.

It should be noted that although various steps of the methods of the present disclosure are depicted in the figures in a particular order, this does not require or imply that the steps must be executed in that particular order, or that all illustrated steps must be executed to achieve a desired result. Additionally or alternatively, certain steps may be omitted, multiple steps may be combined into one step for execution, and/or one step may be decomposed into multiple steps for execution, and the like.

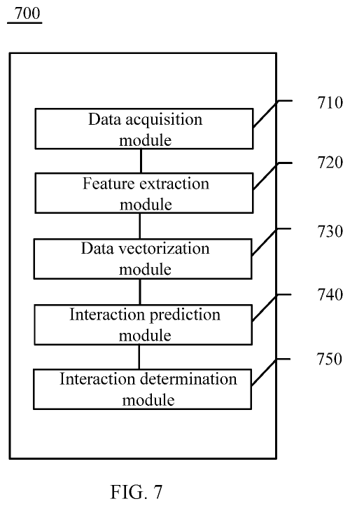

In some exemplary embodiments, an apparatus for predicting an RNA-protein interaction is also provided. The apparatus can be applied to a server or a terminal device. Referring to FIG. 7, the apparatus 700 for predicting the RNA-protein interaction may include a data acquisition module 710, a feature extraction module 720, a data vectorization module 730, an interaction prediction module 740, and an interaction determination module 750.

The data acquisition module 710 is configured to acquire an RNA-protein pair to be predicted.

The feature extraction module 720 is configured to obtain a sequence feature of the RNA-protein pair to be predicted by performing feature extraction on the RNA-protein pair to be predicted.

The data vectorization module 730 is configured to obtain an RNA sequence representation vector and a protein sequence representation vector in the RNA-protein pair to be predicted by vectorizing the RNA-protein pair to be predicted.

The interaction prediction module 740 is configured to obtain respectively by using multiple interaction prediction models, multiple interaction prediction values of the RNA-protein pair to be predicted, based on the sequence feature of the RNA-protein pair to be predicted, the RNA sequence representation vector and the protein sequence representation vector in the RNA-protein pair to be predicted.

The interaction determination module 750 is configured to determine an interaction between the RNA and the protein according to the multiple interaction prediction values.

In some embodiments, the feature extraction module 720 includes a feature set acquisition module and a feature determination module.

The feature set acquisition module is configured to obtain an original sequence feature set.

The feature determination module is configured to determine a sequence feature of the RNA-protein pair to be predicted according to the original sequence feature set.

In some embodiments, the feature determination module includes a sequence conversion unit and a first sequence search unit.

The sequence conversion unit is configured to convert an RNA sequence and a protein sequence in the RNA-protein pair to be predicted into k-mer subsequences, respectively.

The first sequence search unit is configured to search for each of the k-mer subsequences in the original sequence feature set, and obtain the sequence feature of the RNA-protein pair to be predicted according to a search result.

In some embodiments, the feature determination module includes a sequence conversion unit, a sequence combination unit and a second sequence search unit.

The sequence conversion unit is configured to convert an RNA sequence and a protein sequence in the RNA-protein pair to be predicted into k-mer subsequences, respectively, herein, the k-mer subsequences comprise an RNA k-mer subsequence and a protein k-mer subsequence.

The sequence combination unit is configured to combine the RNA k-mer subsequence and the protein k-mer subsequence to obtain multiple RNA-protein k-mer subsequence pairs.

The second sequence search unit is configured to search for each of the RNA-protein k-mer subsequence pairs in the original sequence feature set, and obtain the sequence feature of the RNA-protein pair to be predicted according to a search result.

In some embodiments, the feature determination module includes a sequence conversion unit, a first sequence search unit, a sequence combination unit, a second sequence search unit and a feature splicing unit.

The sequence conversion unit is configured to convert an RNA sequence and a protein sequence in the RNA-protein pair to be predicted into k-mer subsequences, respectively, herein, the k-mer subsequences comprise an RNA k-mer subsequence and a protein k-mer subsequence.

The first sequence search unit is configured to search for each of the k-mer subsequences in the original sequence feature set to obtain a first sequence feature.

The sequence combination unit is configured to combine the RNA k-mer subsequence and the protein k-mer subsequence to obtain multiple RNA-protein k-mer subsequence pairs.

The second sequence search unit is configured to search for each of the RNA-protein k-mer subsequence pairs in the original sequence feature set to obtain a second sequence feature.

The feature splicing unit is configured to form the sequence feature of the RNA-protein pair to be predicted by using the first sequence feature and the second sequence feature.

In some embodiments, the feature set acquisition module includes a data set acquisition module and a feature extraction module.

The data set acquisition module is configured to obtain an original data set.

The feature extraction module is configured to perform feature extraction on each RNA-protein pair in the original data set to obtain the original sequence feature set.

In some embodiments, the feature extraction module includes a sequence generation unit, a variance calculation unit and a data set determination unit.

The sequence generation unit is configured to obtain k-mer subsequences by performing permutation with repetition on basic units of the RNA and the protein.

The variance calculation unit is configured to count frequency of occurrence of each of the k-mer subsequences in the original data set, and calculate variance of each of the k-mer subsequences according to the frequency of occurrence.

The data set determination unit is configured to determine the original sequence feature set according to the variance of each of the k-mer subsequences.

In some embodiments, the variance calculation unit includes a number counting subunit, a frequency calculation subunit, a sequence marking subunit, and a variance calculation subunit.

The number counting subunit is configured to count number of occurrence of each of the k-mer subsequences in the original data set.

The frequency calculation subunit is configured to calculate the frequency of occurrence of each of the k-mer subsequences in the original data set according to the number of occurrence.

The sequence marking subunit is configured to mark whether each of the k-mer subsequences occurs in each RNA-protein pair by traversing the original data set.

The variance calculation subunit is configured to calculate the variance of each of the k-mer subsequences according to the frequency of occurrence of each of the k-mer subsequences in the original data set and a marking value of each of the k-mer subsequences in each RNA-protein pair.

In some embodiments, the variance calculation subunit is configured to calculate the variance Varᵢ of an i^{th} k-mer subsequence according to:

$${Var}_{i} = {\sum }_{n = 1}^{n = N} {( {Appear}_{{i}_{n}} - {Freq}_{i} )}^{2} ,$$

where Appearᵢ_{ₙ} is the marking value of the i^{th} k-mer subsequence in the n^{th} RNA-protein pair, Freqᵢ is the frequency of occurrence of the i^{th} k-mer subsequence in the original data set, and Nis a total number of RNA-protein pairs in the original data set.

In some embodiments, the data set determination unit is configured to determine a k-mer subsequence that meets a preset condition according to the variance of each of the k-mer subsequences, and form the original sequence feature set by using the k-mer subsequence that meets the preset condition.

In some embodiments, the feature extraction module further includes a first itemset generation unit, a frequent itemset generation unit, a second itemset generation unit, a support degree determination unit, and a feature set acquisition unit.

The first itemset generation unit is configured to convert an RNA sequence and a protein sequence in each RNA-protein pair into k-mer subsequences, respectively, and form a first candidate itemset by using the k-mer subsequences, herein the k-mer subsequences comprise an RNA k-mer subsequence and a protein k-mer subsequence.

The frequent itemset generation unit is configured to count frequency of occurrence of each of the k-mer subsequences contained in the first candidate itemset in the original data set, and form a frequent itemset by using a k-mer subsequence that meets a preset occurrence frequency threshold.

The second itemset generation unit is configured to cross-combine the RNA k-mer subsequence and the protein k-mer subsequence contained in the frequent itemset, and form a second candidate itemset by using a k-mer subsequence pair obtained through cross-combination.

The support degree determination unit is configured to count frequency of occurrence of each k-mer subsequence pair contained in the second candidate itemset in the original data set, to obtain a support degree of each k-mer subsequence pair.

The feature set acquisition unit is configured to form the original sequence feature set by using a k-mer subsequence pair whose support degree meets a preset condition.

In some embodiments, the data vectorization module 730 includes a sequence conversion unit, a first vectorization unit, a first splicing unit, a second vectorization unit, and a second splicing unit.

The sequence conversion unit is configured to convert an RNA sequence and a protein sequence in the RNA-protein pair to be predicted into k-mer subsequences, respectively, herein the k-mer subsequences comprise M RNA k-mer subsequence and N protein k-mer subsequence.

The first vectorization unit is configured to vectorize each of the M RNA k-mer subsequences to obtain M RNA k-mer vectors.

The first splicing unit is configured to obtain the RNA sequence representation vector by splicing the M RNA k-mer vectors.

The second vectorization unit is configured to vectorize each of the N protein k-mer subsequences to obtain N protein k-mer vectors.

The second splicing unit is configured to obtain the protein sequence representation vector by splicing the N protein k-mer vectors.

In some embodiments, the interaction prediction module 740 includes a first prediction unit and a second prediction unit.

The first prediction unit is configured to input the sequence feature of the RNA-protein pair to be predicted into at least one first interaction prediction model to obtain at least one first interaction prediction value.

The second prediction unit is configured to input the RNA sequence representation vector and the protein sequence representation vector in the RNA-protein pair to be predicted into at least one second interaction prediction model to obtain at least one second interaction prediction value.

In some embodiments, the interaction prediction module 740 includes a third prediction unit and a fourth prediction unit.

The third prediction unit is configured to input the sequence feature of the RNA-protein pair to be predicted into at least one traditional machine learning model to obtain at least one first interaction prediction value.

The fourth prediction unit is configured to input the RNA sequence representation vector and the protein sequence representation vector in the RNA-protein pair to be predicted into at least one deep learning model to obtain at least one second interaction prediction value.

In some embodiments, each deep learning model includes at least two sub-deep learning models, and the fourth prediction unit includes a first feature generation unit, a second feature generation unit, and a feature fusion unit.

The first feature generation unit is configured to input the RNA sequence representation vector in the RNA-protein pair to be predicted into a first sub-deep learning model to obtain a first sequence feature.

The second feature generation unit is configured to input the protein sequence representation vector in the RNA-protein pair to be predicted into a second sub-deep learning model to obtain a second sequence feature.

The feature fusion unit is configured to fuse the first sequence feature and the second sequence feature, and obtain the second interaction prediction value according to a fused feature.

In some embodiments, the traditional machine learning model comprises at least one of a logistic regression model, a support vector machine model and a decision tree model, and the deep learning model comprises at least one of a convolutional neural network model and a recurrent neural network model.

In some embodiments, the interaction determination module 750 includes a weighted calculation unit and an interaction determination unit.

The weighted calculation unit is configured to calculate a weighted sum of the multiple interaction prediction values.

The interaction determination unit is configured to determine the interaction between the RNA and the protein according to a calculation result.

In some embodiments, the interaction determination unit includes a first interaction determination subunit and a second interaction determination subunit.

The first interaction determination subunit is configured to determine the interaction between the RNA and the protein occurs in response to the calculation result being greater than a preset interaction prediction threshold.

The second interaction determination subunit is configured to determine the interaction between the RNA and the protein does not occur in response to the calculation result being less than or equal to the preset interaction prediction threshold.

In some embodiments, the apparatus 700 for predicting an RNA-protein interaction further includes a joint training module.

The joint training module is configured to train the multiple interaction prediction models jointly.

In some embodiments, the joint training module includes a training data acquisition unit, a first prediction value output unit, a second prediction value output unit, a loss value calculation unit, and a model parameter adjustment unit.

The training data acquisition unit is configured to obtain a training data set, herein the training data set comprises a positive RNA-protein pair and a negative RNA-protein pair.

The first prediction value output unit is configured to obtain multiple interaction prediction values for each RNA-protein pair contained in the training data set by using the multiple interaction prediction models, respectively.

The second prediction value output unit is configured to obtain a joint prediction value for each RNA-protein pair contained in the training data set according to the multiple interaction prediction values.

The loss value calculation unit is configured to perform calculation on the joint prediction value and a marking value for each RNA-protein pair contained in the training data set by using a loss function to obtain a corresponding loss value.

The model parameter adjustment unit is configured to adjust model parameters of the multiple interaction prediction models according to the loss value.

In some embodiments, the second prediction value output unit includes a second prediction value output subunit.

The second prediction value output subunit is configured to obtain the joint prediction value for each RNA-protein pair contained in the training data by calculating a weighted sum of the multiple interaction prediction values for each RNA-protein pair contained in the training data set.

In some embodiments, the second prediction value output subunit is configured to calculating the joint prediction value yₒᵤₜ for each RNA-protein pair contained in the training data according to:

$${y}_{out} = α * {y}_{1} + β * {y}_{2} + γ * {y}_{3} ,$$

where y₁ is an output of the traditional machine learning model, y₂ is an output of the convolutional neural network model, y₃ is an output of the recurrent neural network model, and α, β, and γ are respectively weight parameters of the traditional machine learning model, the convolutional neural network model and the recurrent neural network model.

In some embodiments, the model parameter adjustment unit is configured to iteratively update the model parameters of the multiple interaction prediction models based on the loss value, and end training of the model parameters of the multiple interaction prediction models in response to satisfaction of an iteration termination condition, to predict the interaction of the RNA-protein pair to be predicted by using the multiple interaction prediction models trained.

In some embodiments, the apparatus 700 for predicting an RNA-protein interaction further includes a data output module.

The data output module is configured to output a prediction result of the interaction between the RNA and the protein.

Specific details of each module in above-mentioned apparatus for predicting an RNA-protein interaction have been described in detail in corresponding embodiments of the method for predicting an RNA-protein interaction, which will not be repeated here.

The modules in above apparatus can be a general purpose processor, including a central processing unit, a network processor, etc. The modules can also be a digital signal processor, an application-specific integrated circuit, a field programmable gate array or other programmable logic devices, discrete gates or transistor logic devices, discrete hardware components. The modules can also be implemented in the form of software, firmware and the like. The processors in above apparatus may be an independent processor, or may be integrated together.

Exemplary embodiments of the present disclosure also provide a computer-readable storage medium on which a program product capable of implementing the method provided by embodiments of the present disclosure described above is stored. In some embodiments, various aspects of the present disclosure can also be implemented in the form of a program product, which includes program codes, and when the program product is run on an electronic device, the program codes are configured to cause the electronic device to execute steps according to various exemplary embodiments of the present disclosure which are described in “example methods” embodiments in the present specification. The program product can be a portable compact disk read only memory (CD-ROM) including program codes, and can be run on a terminal device, such as running on a personal computer. However, the program product of the present disclosure is not limited thereto. In the present disclosure, a readable storage medium may be any tangible medium that contains or stores a program, and the program can be used by or in conjunction with an instruction execution system, apparatus, or device.

The program product may be any combination of one or more readable media. The readable medium may be a readable signal medium or a readable storage medium. The readable storage medium may be, for example, but not limited to, an electrical, magnetic, optical, electromagnetic, infrared, or semiconductor system, apparatus or device, or a combination of any of the above. More specific examples (non-exhaustive list) of readable storage media include, electrical connections with one or more wires, portable disks, hard disks, random access memory (RAM), read only memory (ROM), erasable programmable read only memory (EPROM or flash), optical fiber, portable compact disk read only memory (CD-ROM), optical storage devices, magnetic storage devices, or any suitable combination of the above.

A computer readable signal medium may include a propagated data signal in a baseband or as part of a carrier wave with readable program codes embodied thereon. Such propagated data signals may take a variety of forms, including but not limited to electromagnetic signals, optical signals, or any suitable combination of the above. A readable signal medium may also be any readable medium other than a readable storage medium that can transmit, propagate, or transport the program used by or in connection with the instruction execution system, apparatus, or device.

Program codes embodied on a readable medium may be transmitted using any suitable medium, including but not limited to wireless, wireline, optical fiber cable, RF, etc., or any suitable combination of the above.

Program codes for carrying out operations of the present disclosure may be written in any combination of one or more programming languages, including object-oriented programming languages, such as Java, C++, etc., as well as conventional procedural programming languages, such as “C” language or similar programming languages. The program codes may be executed entirely on a user's computing device, partly on the user's device, as a stand-alone software package, partly on the user's computing device and partly on a remote computing device, or entirely on the remote computing device or a server. Where the remote computing device is involved, the remote computing device may be connected to the user's computing device over any kind of network, including a local area network (LAN) or a wide area network (WAN), or may be connected to an external computing device (e.g., connected via the Internet with the help of an Internet Service Provider).

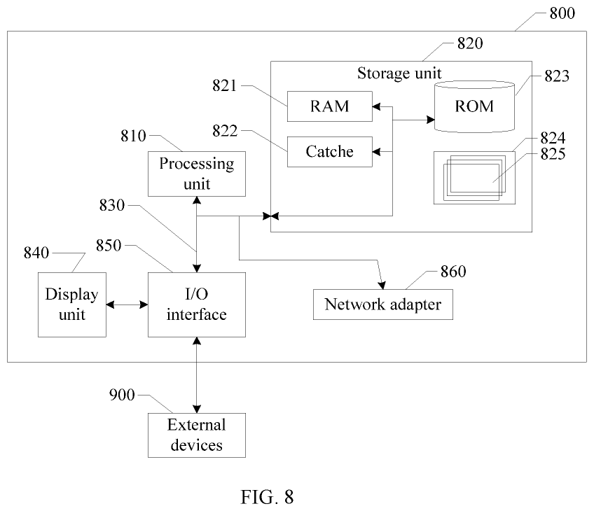

Exemplary embodiments of the present disclosure also provide an electronic device capable of implementing above method. An electronic device 800 according to exemplary embodiments of the present disclosure is described below with reference to FIG. 8. The electronic device 800 shown in FIG. 8 is only an example, and should not impose any limitation on the function and a scope of use of embodiments of the present disclosure.

As shown in FIG. 8, the electronic device 800 is embodied in the form of a general-purpose computing device. Components of the electronic device 800 may include, but not limited to at least one processing unit 810, at least one storage unit 820, a bus 830 connecting different system components (including the storage unit 820 and the processing unit 810), and a display unit 840.

The storage unit 820 stores program codes, and the program codes can be executed by the processing unit 810, so that the processing unit 810 executes steps according to various exemplary embodiments of the present disclosure that are described in the “example methods” section in this specification. For example, the processing unit 810 may execute steps of any one or multiple methods shown in FIGS. 2 to 6.

The storage unit 820 may include a readable medium in the form of a volatile storage unit, such as a random access storage unit (RAM) 821 and/or a cache storage unit 822, and may further include a read only storage unit (ROM) 823.

The storage unit 820 may further include a program/utility 824 having a set of (at least one) program modules 825, and such program modules 825 includes but not limited to an operating system, one or more application programs, other program modules, and program data. Each or some combination of these examples may include an implementation of a network environment.

The bus 830 may be representative of one or more of several types of bus structures, including a memory cell bus or a memory cell controller, a peripheral bus, a graphics acceleration port, a processing unit, or a local area bus using any of a variety of bus structures.

The electronic device 800 may also communicate with one or more external devices 900 (e.g., keyboards, pointing devices, Bluetooth devices, etc.), as well as with one or more devices that enable a user to communicate with the electronic device 800, and/or communicate with any device (e.g., router, modem, etc.) that enables the electronic device 800 can communicate with one or more other computing devices. Such communication may be conducted through input/output (I/O) interface 850. Also, the electronic device 800 may communicate with one or more networks (e.g., a local area network (LAN), a wide area network (WAN), and/or a public network such as the Internet) through a network adapter 860. As is shown, the network adapter 860 communicates with other modules of electronic device 800 via bus 830. It should be understood that, although not shown, other hardware and/or software modules may be used in conjunction with electronic device 800, including but not limited to microcodes, device drivers, redundant processing units, external disk drive arrays, RAID systems, tape drivers, data backup storage systems, etc.

In some embodiments, the method for predicting an RNA-protein interaction described in the present disclosure may be performed by the processing unit 810 of the electronic device. In some embodiments, the RNA-protein pair to be predicted/the RNA sequence to be predicted/the protein sequence to be predicted, the original data set, and the training data set for training each interaction prediction model, etc., can be input through the input interface 850. For example, the RNA-protein pair to be predicted, the original data set, and the training data set for training each interaction prediction model, etc., are input through the user interface of the electronic device. In some embodiments, the prediction result of the interaction of the RNA-protein pair to be predicted can be output to the external device 900 through the output interface 850 for the user to view.

From the descriptions of the above embodiments, it can be easily understood for those skilled in the art that the exemplary embodiments described herein may be implemented by software, or by a combination of software and necessary hardware. Therefore, the technical solutions according to the embodiments of the present disclosure may be embodied in the form of software products. The software products may be stored in a non-volatile storage medium (which may be CD-ROM, U disk, mobile hard disk, etc.) or on the network, including several instructions to cause a computing device (which may be a personal computer, a server, a mobile terminal, or a network device, etc.) to implement the method according to the embodiments of the present disclosure.

Furthermore, the above-mentioned figures are merely schematic illustrations of the processes included in the methods according to the exemplary embodiments of the present disclosure, and are not intended to restrict. It is easy to understand that the processes shown in the above figures do not indicate or restrict the chronological order of these processes. In addition, it is also readily understood that these processes may be executed synchronously or asynchronously, for example, in multiple modules.

It should be noted that although several modules or units of the apparatus for perform actions are mentioned in the above detailed descriptions, such division is not mandatory. Indeed, according to embodiments of the present disclosure, the features and functions of two or more modules or units described above may be embodied in one module or unit. Conversely, the features and functions of one module or unit described above may be further divided into multiple modules or units to be embodied.

It should be understood that the present disclosure is not limited to the precise structures described above and illustrated in the accompanying drawings, and that various modifications and changes may be made without departing from the scope thereof. The scope of the present disclosure is limited only by the appended claims.

1. A method for predicting an RNA-protein interaction, comprising:
acquiring an RNA-protein pair to be predicted;
obtaining a sequence feature of the RNA-protein pair to be predicted by performing feature extraction on the RNA-protein pair to be predicted;
obtaining an RNA sequence representation vector and a protein sequence representation vector in the RNA-protein pair to be predicted by vectorizing the RNA-protein pair to be predicted;
obtaining respectively by using multiple interaction prediction models, multiple interaction prediction values of the RNA-protein pair to be predicted, based on the sequence feature of the RNA-protein pair to be predicted, the RNA sequence representation vector and the protein sequence representation vector in the RNA-protein pair to be predicted; and
determining an interaction between the RNA and the protein according to the multiple interaction prediction values.

2. The method for predicting an RNA-protein interaction according to claim 1, wherein said obtaining a sequence feature of the RNA-protein pair to be predicted by performing feature extraction on the RNA-protein pair to be predicted comprises:
obtaining an original sequence feature set; and
determining a sequence feature of the RNA-protein pair to be predicted according to the original sequence feature set.

3. The method for predicting an RNA-protein interaction according to claim 2, wherein said determining a sequence feature of the RNA-protein pair to be predicted according to the original sequence feature set comprises:
converting an RNA sequence and a protein sequence in the RNA-protein pair to be predicted into k-mer subsequences, respectively; and
searching for each of the k-mer subsequences in the original sequence feature set, and obtaining the sequence feature of the RNA-protein pair to be predicted according to a search result.

4. The method for predicting an RNA-protein interaction according to claim 2, wherein said determining a sequence feature of the RNA-protein pair to be predicted according to the original sequence feature set comprises:
converting an RNA sequence and a protein sequence in the RNA-protein pair to be predicted into k-mer subsequences, respectively, wherein the k-mer subsequences comprise an RNA k-mer subsequence and a protein k-mer subsequence;
combining the RNA k-mer subsequence and the protein k-mer subsequence to obtain multiple RNA-protein k-mer subsequence pairs; and
searching for each of the RNA-protein k-mer subsequence pairs in the original sequence feature set, and obtaining the sequence feature of the RNA-protein pair to be predicted according to a search result.

5. The method for predicting an RNA-protein interaction according to claim 2, wherein said determining a sequence feature of the RNA-protein pair to be predicted according to the original sequence feature set comprises:
converting an RNA sequence and a protein sequence in the RNA-protein pair to be predicted into k-mer subsequences, respectively, wherein the k-mer subsequences comprise an RNA k-mer subsequence and a protein k-mer subsequence;
searching for each of the k-mer subsequences in the original sequence feature set to obtain a first sequence feature;
combining the RNA k-mer subsequence and the protein k-mer subsequence to obtain multiple RNA-protein k-mer subsequence pairs;
searching for each of the RNA-protein k-mer subsequence pairs in the original sequence feature set to obtain a second sequence feature; and
forming the sequence feature of the RNA-protein pair to be predicted by using the first sequence feature and the second sequence feature.

6. The method for predicting an RNA-protein interaction according to claim 2, wherein said obtaining an original sequence feature set comprises:
obtaining an original data set; and
performing feature extraction on each RNA-protein pair in the original data set to obtain the original sequence feature set.

7. The method for predicting an RNA-protein interaction according to claim 6, wherein said performing feature extraction on each RNA-protein pair in the original data set to obtain the original sequence feature set comprises:
obtaining k-mer subsequences by performing permutation with repetition on basic units of the RNA and the protein, respectively;
counting frequency of occurrence of each of the k-mer subsequences in the original data set, and calculating variance of each of the k-mer subsequences according to the frequency of occurrence; and
determining the original sequence feature set according to the variance of each of the k-mer subsequences.

8. The method for predicting an RNA-protein interaction according to claim 7, wherein said counting frequency of occurrence of each of the k-mer subsequences in the original data set, and calculating variance of each of the k-mer subsequences according to the frequency of occurrence comprises:
counting number of occurrence of each of the k-mer subsequences in the original data set;
calculating the frequency of occurrence of each of the k-mer subsequences in the original data set according to the number of occurrence;
marking whether each of the k-mer subsequences occurs in each RNA-protein pair by traversing the original data set; and
calculating the variance of each of the k-mer subsequences according to the frequency of occurrence of each of the k-mer subsequences in the original data set and a marking value of each of the k-mer subsequences in each RNA-protein pair.

9. The method for predicting an RNA-protein interaction according to claim 8, wherein said calculating the variance of each of the k-mer subsequences according to the frequency of occurrence of each of the k-mer subsequences in the original data set and a marking value of each of the k-mer subsequences in each RNA-protein pair comprises calculating the variance Varᵢ of an i^{th} k-mer subsequence according to:
$${Var}_{i} = {\sum }_{n = 1}^{n = N} {( {Appear}_{{i}_{n}} - {Freq}_{i} )}^{2} ,$$
where Appearᵢ_{ₙ} is the marking value of the i^{th} k-mer subsequence in the n^{th} RNA-protein pair, Freqᵢ is the frequency of occurrence of the i^{th} k-mer subsequence in the original data set, and Nis a total number of RNA-protein pairs in the original data set.

10. The method for predicting an RNA-protein interaction according to claim 7, wherein said determining the original sequence feature set according to the variance of each of the k-mer subsequences comprises:
determining a k-mer subsequence that meets a preset condition according to the variance of each of the k-mer subsequences, and forming the original sequence feature set by using the k-mer subsequence that meets the preset condition.

11. The method for predicting an RNA-protein interaction according to claim 6, wherein said performing feature extraction on each RNA-protein pair in the original data set to obtain the original sequence feature set further comprises:
converting an RNA sequence and a protein sequence in each RNA-protein pair into k-mer subsequences, respectively, and forming a first candidate itemset by using the k-mer subsequences, wherein the k-mer subsequences comprise an RNA k-mer subsequence and a protein k-mer subsequence;
counting frequency of occurrence of each of the k-mer subsequences contained in the first candidate itemset in the original data set, and forming a frequent itemset by using a k-mer subsequence that meets a preset occurrence frequency threshold;
cross-combining the RNA k-mer subsequence and the protein k-mer subsequence contained in the frequent itemset, and forming a second candidate itemset by using a k-mer subsequence pair obtained through cross-combination;
counting frequency of occurrence of each k-mer subsequence pair contained in the second candidate itemset in the original data set, to obtain a support degree of each k-mer subsequence pair; and
forming the original sequence feature set by using a k-mer subsequence pair whose support degree meets a preset condition.

12. The method for predicting an RNA-protein interaction according to claim 1, wherein said obtaining an RNA sequence representation vector and a protein sequence representation vector in the RNA-protein pair to be predicted by vectorizing the RNA-protein pair to be predicted comprises:
converting an RNA sequence and a protein sequence in the RNA-protein pair to be predicted into k-mer subsequences, respectively, wherein the k-mer subsequences comprise M RNA k-mer subsequence and N protein k-mer subsequence;
vectorizing each of the M RNA k-mer subsequences to obtain M RNA k-mer vectors;
obtaining the RNA sequence representation vector by splicing the M RNA k-mer vectors;
vectorizing each of the N protein k-mer subsequences to obtain N protein k-mer vectors; and
obtaining the protein sequence representation vector by splicing the N protein k-mer vectors.

13. The method for predicting an RNA-protein interaction according to claim 1, wherein said obtaining respectively by using multiple interaction prediction models, multiple interaction prediction values of the RNA-protein pair to be predicted, based on the sequence feature of the RNA-protein pair to be predicted, the RNA sequence representation vector and the protein sequence representation vector in the RNA-protein pair to be predicted comprises:
inputting the sequence feature of the RNA-protein pair to be predicted into at least one first interaction prediction model to obtain at least one first interaction prediction value; and
inputting the RNA sequence representation vector and the protein sequence representation vector in the RNA-protein pair to be predicted into at least one second interaction prediction model to obtain at least one second interaction prediction value.

14. The method for predicting an RNA-protein interaction according to claim 1, wherein said obtaining respectively by using multiple interaction prediction models, multiple interaction prediction values of the RNA-protein pair to be predicted, based on the sequence feature of the RNA-protein pair to be predicted, the RNA sequence representation vector and the protein sequence representation vector in the RNA-protein pair to be predicted comprises:
inputting the sequence feature of the RNA-protein pair to be predicted into at least one traditional machine learning model to obtain at least one first interaction prediction value; and
inputting the RNA sequence representation vector and the protein sequence representation vector in the RNA-protein pair to be predicted into at least one deep learning model to obtain at least one second interaction prediction value.

15. The method for predicting an RNA-protein interaction according to claim 14, wherein each of the at least one deep learning model comprises at least two sub-deep learning models; and said inputting the RNA sequence representation vector and the protein sequence representation vector in the RNA-protein pair to be predicted into at least one deep learning model to obtain at least one second interaction prediction value comprises:
inputting the RNA sequence representation vector in the RNA-protein pair to be predicted into a first sub-deep learning model to obtain a first sequence feature;
inputting the protein sequence representation vector in the RNA-protein pair to be predicted into a second sub-deep learning model to obtain a second sequence feature; and
fusing the first sequence feature and the second sequence feature, and obtaining the second interaction prediction value according to a fused feature.

16. The method for predicting an RNA-protein interaction according to claim 14, wherein the traditional machine learning model comprises at least one of a logistic regression model, a support vector machine model and a decision tree model, and the deep learning model comprises at least one of a convolutional neural network model and a recurrent neural network model.

17. The method for predicting an RNA-protein interaction according to claim 1, wherein said determining an interaction between the RNA and the protein according to the multiple interaction prediction values comprises:
calculating a weighted sum of the multiple interaction prediction values; and
determining the interaction between the RNA and the protein according to a calculation result.

18. The method for predicting an RNA-protein interaction according to claim 17, wherein said determining the interaction between the RNA and the protein according to a calculation result comprises:
determining the interaction between the RNA and the protein occurs in response to the calculation result being greater than a preset interaction prediction threshold; and
determining the interaction between the RNA and the protein does not occur in response to the calculation result being less than or equal to the preset interaction prediction threshold.

19-23. (canceled)

24. The method for predicting an RNA-protein interaction according to claim 1, further comprising:
outputting a prediction result of the interaction between the RNA and the protein.

25-26. (canceled)

27. An electronic device, comprising:
a processor; and
a memory for storing instructions executable by the processor;
wherein the processor is configured to:
acquire an RNA-protein pair to be predicted;
obtain a sequence feature of the RNA-protein pair to be predicted by performing feature extraction on the RNA-protein pair to be predicted;
obtain an RNA sequence representation vector and a protein sequence representation vector in the RNA-protein pair to be predicted by vectorizing the RNA-protein pair to be predicted;
obtain respectively by using multiple interaction prediction models, multiple interaction prediction values of the RNA-protein pair to be predicted, based on the sequence feature of the RNA-protein pair to be predicted, the RNA sequence representation vector and the protein sequence representation vector in the RNA-protein pair to be predicted; and
determine an interaction between the RNA and the protein according to the multiple interaction prediction values.


SEQUENCE LISTING (2 sequences)

SEQ ID NO: 1 - protein, 6 aa, Artificial
  Exemplatory sequence
ALQDVG

SEQ ID NO: 2 - RNA, 10 nt, Artificial
  Exemplatory sequence
aaaacccggg